(19) United States
(12) Patent Application Publication
(10) Pub. No.: US 2006/0057209 A1
(43) Pub. Date: Mar. 16, 2006

(54) Methods, compositions and devices, including microfluidic devices, comprising coated hydrophobic surfaces

(75) Inventors: Robert G. Chapman, San Mateo, CA (US); Mingqi Zhao, San Jose, CA (US); Jing Ni, Sunnyvale, CA (US); Luc J. Bousse, Los Altos, CA (US); John T. Stults, Redwood City, CA (US); Say Yang, Daly City, CA (US)

Correspondence Address: WILSON SONSINI GOODRICH & ROSATI, 650 PAGE MILL ROAD, PALO ALTO, CA, 94304-1050, US

(73) Assignee: Predicant Biosciences, Inc., South San Francisco, CA (US)

(21) Appl. No.: 10/942,612

(22) Filed: Sep. 16, 2004

(51) Int. Cl. A61K 9/14

(52) U.S. Cl. 424/486

(57) ABSTRACT

Methods are disclosed for coating at least a portion of a hydrophobic surface, including the surfaces of plastics or other polymers. Such methods include the use of a first coating layer and/or region that interacts with the hydrophobic surface, although the formation of a chemical bond between the first coating layer and the hydrophobic surface is not required. Subsequent layers may then interact chemically or non-chemically with at least a portion of the first coating layer and/or region. Such coated surfaces may be part of a device or apparatus, including microfluidic devices.

### BACKGROUND OF THE INVENTION

Many materials have at least one hydrophobic surface. Examples include the surfaces of plastics and other polymeric materials. These hydrophobic surfaces can be present on or components of a device or apparatus. However, the requirements of the device or apparatus may dictate modification of at least one property of at least a portion of such hydrophobic surfaces. Many types of modifications can be envisioned; by way of example only, it might be desirable to decrease the hydrophobicity of the surface or to enhance the ionic content of the surface. One way to accomplish this modification would be to add at least one additional material in or onto (i.e., coat) at least a portion of the hydrophobic surface. Multiple materials may be added to create more complex surfaces or surfaces with properties tuned to a user's needs. Generally, such coatings should be stable and/or the stability controllable by the fabricator or user of the device or apparatus.

### SUMMARY OF THE INVENTION

Presented herein are methods for adding another material in or onto, that is, coat, at least a portion of a hydrophobic surface. Also presented herein, are surfaces on or in which another material has been coated so that the properties of the original surface has been modified. Further presented are devices comprising at least one surface that has been coated, at least in part, with another material so that the properties of the original surface has been modified. Also presented are methods for making and using devices that comprise at least one surface on or in which another material has been coated. Further presented are multi-channel microfluidic devices in which at least two channels comprise differently coated surfaces. Also presented is the application of the coated microfluidic devices for the separation and analysis of biological samples.

In one aspect is a surface comprising the structure S/A/Z, wherein S is selected from the group consisting of a hydrophobic surface, a covalently modified hydrophobic surface and a functionalized hydrophobic surface, A is an amphiphilic region comprising a monolayer of an amphiphilic polymer or a modified amphiphilic polymer, and Z is a charged region comprising a monolayer of a non-amphiphilic charged polymer or a modified non-amphiphilic charged polymer; wherein the interaction between S and A comprises hydrophobic interactions and/or covalent bonds, and the interaction between A and Z comprises electrostatic and/or covalent bonds. In one embodiment, the amphiphilic polymer or modified amphiphilic polymer is no more than a monolayer. In a further embodiment, the charged polymer or modified charged polymer is no more than a monolayer.

In a further embodiment of the aforementioned aspect, S is a hydrophobic surface comprising a hydrophobic polymer. In further embodiments, the amphiphilic polymer or modified amphiphilic polymer is no more than a monolayer. In yet further embodiment, the charged polymer or modified charged polymer is no more than a monolayer. In a further embodiment, the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof. In still a further embodiment, the hydrophobic surface comprises a mixture or blend of at least two hydrophobic polymers. In further embodiments, the hydrophobic polymer is a methacrylate polymer or the hydrophobic polymer is polycarbonate.

In a further embodiment of the aforementioned aspect, S is a modified hydrophobic surface comprising a modified hydrophobic polymer. In a further embodiment, the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof. In further embodiments, the hydrophobic polymer is a modified methacrylate polymer or the hydrophobic polymer is modified polycarbonate. In any of these embodiments, the modification can be a covalent modification and/or a partial modification.

Such modified hydrophobic polymers may be made by a method comprising exposing a hydrophobic polymer surface with a nucleophile and/or exposing a hydrophobic polymer surface with an electrophile. Further, in such methods, the exposing step may be sufficient to partially modify the hydrophobic polymer surface. Further, in such methods, the hydrophobic polymer surface may be either a methacrylate surface or a polycarbonate surface.

In any of the surfaces comprising the structure S/A/Z, A may comprise an amphiphilic polymer or a modified amphiphilic polymer. In further embodiments, the amphiphilic polymer comprises a moiety selected from the group consisting of an aryl, an alkyl, and a halogenated alkyl. In still further embodiments, the modified amphiphilic polymer comprises a moiety selected from the group consisting of an aryl, an alkyl, and a halogenated alkyl. In still further embodiments, the amphiphilic polymer comprises polystyrene units. In yet still further embodiments, the modified amphiphilic polymer comprises polystyrene units. In still further embodiments, the amphiphilic polymer comprises positively charged moieties or the amphiphilic polymer comprises negatively charged moieties. In yet still further embodiments, the amphiphilic polymer comprises maleic anhydride units or the amphiphilic polymer is derived from maleic anhydride units.

The amphiphilic region described above may be made by a method comprising reacting a non-amphiphilic polymer with at least one nucleophile to form an amphiphilic polymer. In further embodiments, the nucleophile is a charged nucleophile or the nucleophile is a neutral nucleophile. In still further embodiments, the method further comprises reacting the non-amphiphilic polymer with an additional nucleophile. In still further embodiments of such methods, at least a portion of the non-amphiphilic polymer is in contact with S prior to the reacting step. In still further embodiments, such methods further comprise exposing the amphiphilic polymer to S. In yet further embodiments, the exposing step is prior to the reacting step or the exposing step is after the reacting step or the exposing step is simultaneous with the reacting step. In still further embodiments, the method further comprises reacting the amphiphilic polymer with an additional reagent thereby forming a modified amphiphilic surface. In still further embodiments of any of these methods, the non-amphiphilic polymer comprises maleic anhydride units.

In still further embodiments of any of these methods, S is a hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof. In further embodiments, the hydrophobic polymer is a methacrylic polymer or the hydrophobic polymer is a polycarbonate polymer. In alternative further embodiments of any of these methods, S is a modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

In any of the surfaces comprising the structure S/A/Z, Z may be a non-amphiphilic charged polymer or Z may be a modified non-amphiphilic charged polymer. In further embodiments, Z comprises negatively-charged moieties or Z comprises positively-charged moieties. In further embodiments, the positively-charged moieties are quarternary amines. In further embodiments, the molecular weight of Z is greater than 20,000 atomic mass units or the molecular weight of Z is greater than 20,000 atomic mass units.

In any of the aforementioned surfaces, Z may be made by a method comprising exposing a surface comprising the structure S/A to non-amphiphilic charged polymer. In still further embodiments, the method further comprises reacting the non-amphiphilic charged polymer with a reagent thereby forming a modified non-amphiphilic charged polymer. In further embodiments, the exposing step is prior to the reacting step.

In another embodiment described herein is a surface comprising the structure S/P/R, wherein S is selected from the group consisting of a hydrophobic surface, a covalently modified hydrophobic surface, and a functionalized hydrophobic surface, P is a functionalized region comprising a monolayer of a linkable hydrophobic polymer or a modified linkable hydrophobic polymer, and R is a charged region comprising a monolayer of a linkable charged hydrophilic polymer or a modified linkable charged hydrophilic polymer; wherein the interaction between S and P comprises hydrophobic interactions and/or covalent bonds, and the interaction between P and R comprises covalent bonds, and/or electrostatic bonds, and/or hydrophobic interactions.

In further embodiments of such surfaces, the linkable hydrophobic polymer or the modified linkable hydrophobic polymer is no more than a monolayer or the linkable charged hydrophilic polymer or modified linkable charged hydrophilic polymer is no more than a monolayer. In further embodiments, S is a hydrophobic surface comprising a hydrophobic polymer. In further embodiments, the linkable hydrophobic polymer or the modified linkable hydrophobic polymer is no more than a monolayer. In still further embodiments, the linkable charged hydrophilic polymer or modified linkable charged hydrophilic polymer is no more than a monolayer.

In still further embodiments of such surfaces, the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof. In still further embodiments, the hydrophobic surface comprises a mixture or blend of at least two hydrophobic polymers. In further embodiments, the hydrophobic polymer is a methacrylate polymer or the hydrophobic polymer is polycarbonate.

In other embodiments of such surfaces, S is a modified hydrophobic surface comprising of a modified hydrophobic polymer. In further embodiments, the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof. In further embodiments, the hydrophobic polymer is a modified methacrylate polymer or the hydrophobic polymer is modified polycarbonate. In further embodiments, the modification is a covalent modification and/or the modification is a partial modification.

Also described are methods for forming the modified hydrophobic polymer in a surface comprising the structure S/P/R, comprising exposing a hydrophobic polymer surface with a nucleophile or exposing a hydrophobic polymer surface with an electrophile. In further embodiments, the exposing step is sufficient to partially modify the hydrophobic polymer surface. In further embodiments, the hydrophobic polymer surface is a methacrylate surface or the hydrophobic polymer surface is a polycarbonate surface.

In further embodiments of a surface having the structure S/P/R, P comprises a linkable hydrophobic polymer or P comprises a modified linkable hydrophobic polymer. In further embodiments, the linkable hydrophobic polymer comprises a moiety selected from the group consisting of an aryl, an alkyl, and a halogenated alkyl or the linkable hydrophobic polymer comprises a moiety selected from the group consisting of a vinyl and a substituted vinyl. In still further embodiments, the modified linkable hydrophobic polymer comprises a moiety selected from the group consisting of an aryl, an alkyl, and a halogenated alkyl or the modified linkable hydrophobic polymer comprises a moiety selected from the group consisting of a vinyl, and a substituted vinyl. In still further embodiments, the linkable hydrophobic polymer comprises poly(1,14-tetradecanediol dimethacrylate) units or the modified linkable hydrophobic polymer comprises poly(1,14-tetradecanediol dimethacrylate) units.

Also described herein are methods of making the functionalized region of surfaces having the structure S/P/R, comprising reacting a non-linkable hydrophobic polymer with at least one nucleophile to form the linkable hydrophobic polymer. In further embodiments, the nucleophile comprises a moiety selected from the group consisting of a vinyl and a substituted vinyl. In other embodiments, the method further comprises reacting the non-linkable hydrophobic polymer with an additional nucleophile. In further embodiments, at least a portion of the non-linkable hydrophobic polymer is in contact with S prior to the reacting step. In other embodiments, the method further comprises, exposing the non-linkable hydrophobic polymer to S prior to the reacting step or exposing the non-linkable hydrophobic polymer to S simultaneous with the reacting step. In a further embodiment, the method comprises exposing reactive monomeric units of the linkable hydrophobic polymer to S; further embodiments comprise polymerizing the reactive units thereby forming the linkable hydrophobic polymer on S. In any of such embodiments, the method may further comprise reacting the linkable hydrophobic polymer with an additional reagent thereby forming a modified linkable hydrophobic surface.

In any of such methods embodiments, S may be a hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof or S may be a modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof. In further embodiments, the hydrophobic polymer is a methacrylic polymer or the hydrophobic polymer is a polycarbonate polymer.

In further embodiments of a surface comprising the structure S/P/R, R is a linkable charged hydrophilic polymer or R is a modified linkable charged hydrophilic polymer. In further embodiments, R comprises negatively-charged moieties or R comprises positively-charged moieties or R comprises moieties with charge equal to zero. In further embodiments, the positively-charged moieties are quarternary amines. In still further embodiments, the molecular weight of R is greater than 20,000 atomic mass units.

In further embodiments of a surface comprising the structure S/P/R, the charged region may be made by a method comprising exposing the linkable charged hydrophilic polymer to the linkable hydrophobic polymer on S, and reacting the linkable charged hydrophilic polymer with at least a portion of the linkable hydrophobic polymer on S. In further embodiments of a surface comprising the structure S/P/R, the charged region may be made by a method comprising exposing monomeric units of the linkable charged hydrophilic polymer to the linkable hydrophobic polymer on S, and reacting the monomeric units of the linkable charged hydrophilic polymer with at least a portion of the linkable hydrophobic polymer on S. In further embodiments of a surface comprising the structure S/P/R, the charged region may be made by a method comprising exposing the modified reactive charged hydrophilic polymer to the reactive hydrophobic polymer on S, and reacting the modified linkable charged hydrophilic polymer with at least a portion of the linkable hydrophobic polymer on S. In further embodiments of a surface comprising the structure S/P/R, the charged region may be made by a method comprising exposing monomeric units of the modified linkable charged hydrophilic polymer to the linkable hydrophobic polymer on S, and polymerizing the monomeric units of the modified linkable charged hydrophilic polymer with at least a portion of the linkable hydrophobic polymer on S.

In further embodiments is a surface comprising the structure S/N, wherein S is selected from the group consisting of a hydrophobic surface, a covalently modified hydrophobic surface, and a functionalized hydrophobic surface, N is a hydrophilic region comprising a monolayer of neutral hydrophilic polymer or a modified neutral hydrophilic polymer; wherein the interaction between S and N comprises physical entrapment of at least a portion of N in S.

In further embodiments, the neutral hydrophilic polymer or a modified neutral hydrophilic polymer is no more than a monolayer. In further embodiments, S is a hydrophobic surface comprising a hydrophobic polymer. In further embodiments, the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof. In still further embodiments, the hydrophobic surface comprises a mixture or blend of at least two hydrophobic polymers. In further embodiments, the hydrophobic polymer is a methacrylate polymer or the hydrophobic polymer is polycarbonate. In alternative embodiments, S is a modified hydrophobic surface comprising a modified hydrophobic polymer. In further embodiments, the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof. In further embodiments, the hydrophobic polymer is a modified methacrylate polymer or the hydrophobic polymer is modified polycarbonate. In further embodiments, the modification is a covalent modification and/or the modification is a partial modification.

Also described are methods for making such a modified hydrophobic polymer comprising exposing a hydrophobic polymer surface with a nucleophile or exposing a hydrophobic polymer surface with an electrophile. In further embodiments, the exposing step is sufficient to partially modify the hydrophobic polymer surface. In further embodiments, the hydrophobic polymer surface is a methacrylate surface or the hydrophobic polymer surface is a polycarbonate surface.

In further embodiments of surfaces comprising the structure S/N, N comprises a neutral hydrophilic polymer or N comprises a modified neutral hydrophilic polymer. In further embodiments, the neutral hydrophilic polymer is selected from the group consisting of a poly(ethylene glycol) derivative, a poly(ethylene oxide) derivative, a cellulose derivatives, and combinations thereof. In further embodiments, the modified hydrophilic polymer is selected from the group consisting of a modified poly(ethylene glycol) derivative, a modified poly(ethylene oxide) derivative, a modified cellulose derivatives, and combinations thereof. In further embodiments, the neutral hydrophilic polymer comprises poly(ethylene glycol) units. In further embodiments, the neutral hydrophilic polymer comprises poly(ethylene oxide) units or the neutral hydrophilic polymer comprises hydroxypropylmethyl cellulose units. In further embodiments, the modified neutral hydrophilic polymer comprises modified poly(ethylene glycol) units or the modified neutral hydrophilic polymer comprises modified poly(ethylene oxide) units or the modified neutral hydrophilic polymer comprises modified hydroxypropylmethyl cellulose units.

Also described are methods for making the neutral regions of surfaces comprising the structure S/N comprising swelling the hydrophobic surface with a solvent, and exposing the swollen hydrophobic surface to the neutral hydrophilic polymer. In further embodiments, such methods further comprise drying the swollen hydrophobic surface sufficient to entrap at least a portion of the neutral hydrophilic polymer within at least a portion of the hydrophobic surface. In further embodiments, such methods further comprise reacting the neutral hydrophilic polymer with a reagent to form a modified neutral hydrophilic polymer.

Also described herein are surfaces having the structure S/C, wherein S is selected from the group consisting of a hydrophobic surface, a covalently modified hydrophobic surface, and a functionalized hydrophobic surface, C is a hydrophilic region comprising a monolayer of a linkable hydrophilic polymer or a linkable modified hydrophilic polymer; wherein the interaction between S and C comprises covalent attachment of at least a portion of C onto S. In further embodiments, the linkable hydrophilic polymer or a linkable modified hydrophilic polymer is no more than a monolayer. In further embodiments, S is a hydrophobic surface comprising a hydrophobic polymer. In further embodiments, the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof. In further embodiments, the hydrophobic surface comprises a mixture or blend of at least two hydrophobic polymers. In a further embodiment, the hydrophobic polymer is a methacrylate polymer or the hydrophobic polymer is polycarbonate or the hydrophobic polymer is poly(styrene-co-maleic anhydride). In an alternative embodiment, S is a modified hydrophobic surface comprising a modified hydrophobic polymer. In a further embodiment, the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof. In further embodiments, the hydrophobic polymer is a modified methacrylate polymer or the hydrophobic polymer is a modified polycarbonate or the hydrophobic polymer is a modified poly(styrene-co-maleic anhydride). In further embodiments, the modification is a covalent modification and/or the modification is a partial modification.

Also described are methods for forming the modified hydrophobic polymer in surfaces having the structure S/C comprising exposing a hydrophobic polymer surface with a nucleophile or exposing a hydrophobic polymer surface with an electrophile. In further embodiments, the exposing step is sufficient to partially modify the hydrophobic polymer surface. In further embodiments, the hydrophobic polymer surface is a methacrylate surface or the hydrophobic polymer surface is a polycarbonate surface.

In further embodiments of surfaces having the structure S/C, C comprises a linkable hydrophilic polymer or C comprises a linkable modified hydrophilic polymer. In further embodiments, the linkable hydrophilic polymer comprises positively charged moieties or the linkable hydrophilic polymer comprises negatively charged moieties or the linkable hydrophilic polymer is neutral. In further embodiments, linkable modified hydrophilic polymer comprises positively charged moieties or the linkable modified hydrophilic polymer comprises negatively charged moieties or the linkable modified hydrophilic polymer is neutral. In further embodiments, the linkable hydrophilic polymer is selected from the group consisting of polysaccharides, such as hydroxypropylmethyl cellulose, hydroxyethylmethyl cellulose, methyl cellulose and dextran; polyethers, such as polyethylene glycol and polyethylene oxide; polyalcohols, such as polyvinyl alcohol, polyglycerols, polyglycydols; polyamides; polyacrylamides; polyacylamide; polydimethylacrylamide; poly-N-hydroxyethylacrylamide; polyduramide; polyacryloxymorpholine; poly-N-methyloxazoline; poly-N-ethyloxazoline; polyvinylpyrrolidone; zwitterionic polymers, such as poly([3-(methacryloylamino)propyl]dimethyl(3-sulfopropyl)ammonium hydroxide), and proteins such as albumin, gelatin and collagen. In still further embodiments, the linkable modified hydrophilic polymer is a modified version of any of the aforementioned linkable hydrophilic polymers.

In further embodiments are methods of making such hydrophilic region comprising exposing the hydrophobic surface or the modified hydrophobic surface with a hydrophilic polymer or a modified hydrophilic polymer comprised of linkable moieties; and reacting the linkable moieties with at least a portion of the hydrophobic surface or the modified hydrophobic surface. In further embodiments, the linkable unit is a nucleophile or the linkable unit is an electrophile or the linkable unit is chlorohydrin.

Also described herein are microfluidic chips for mass spectrometric analysis comprising a microfluidic body layer formed with a plurality of fluid reservoirs; at least one separation channel and/or at least one side channel that are formed along a length of the microfluidic body layer in fluid communication with at least one fluid reservoir; wherein at least one of the separation channels and/or side channels comprises a charged polymer monolayer coated on a hydrophobic surface; and a cover plate for enclosing the separation channel and the side channel to provide a stable electrospray from the microfluidic chip. In further embodiments, the side channel provides electrical contact to the separation channel or the side channel provides sheath flow. In further embodiments, the charged coating of the side channel is a negatively charged coating, and the separation channel includes a positively charged coating. Such a charged coating may be made using any of the methods described herein. In further embodiments, the charged coating of the side channel is a negatively charged coating, and the separation channel is without a coating. In such methods, the negatively charged coating is produced using any of the methods described herein.

In further embodiments, the charged coating of the side channel is a negatively charged coating, and the separation channel includes a neutral uncharged coating. In further embodiments, such a negatively charged coating is produced using any of the methods described herein, and the neutral uncharged coating is further produced using any of the methods described herein. In a further embodiment, the charged coating of the side channel is a positively charged coating, and the separation channel includes a negatively charged coating. In such embodiments, each of the charged coatings may also be produced using any of the methods described herein. In further embodiments, the charged coating of the side channel is a positively charged coating, and the separation channel is without a coating. In such embodiments, the positively charged coating may be further produced using any of the methods described herein. In further embodiments, the charged coating of the side channel is a positively charged coating, and the separation channel includes a neutral uncharged coating. In such embodiments, the positively charged coating may be further produced using any of the methods described herein and the neutral uncharged coating may be further produced using any of the methods described herein.

In further embodiments, side channel is without a coating, and the separation channel includes a positively charged coating. In such embodiments, the positively charged coating may be further produced using any of the methods described herein. In further embodiments, the side channel is without a coating, and the separation channel includes a negatively charged coating. In such embodiments, the negatively charged coating may be further produced using any of the methods described herein. In further embodiments, the side channel is includes a neutral coating, and the separation channel includes a positively charged coating. In such embodiments, the neutral uncharged coating may be further produced using any of the methods described herein and the positively charged coating may be further produced using any of the methods described herein.

In further embodiments, the side channel is includes a neutral coating, and the separation channel includes a negatively charged coating. In such embodiments, the neutral uncharged coating may be further produced using any of the methods described herein, and the negatively charged coating may be further produced using any of the methods described herein.

In further embodiments of such microfluidic chips, the microfluidic chips further comprise a plurality of electrodes positioned in each fluid reservoir to apply voltages to impart movement of materials within the separation channel and the side channel. In further embodiments, the cover plate extends beyond the microfluidic body layer to form an open-ended distal tip portion at which the separation channel and the side channel terminate to provide an electrospray ionization tip that directs a stable electrospray from the microfluidic chip. In still further embodiments, at least a portion of the open-ended distal tip portion is covered with a hydrophilic material. In still further embodiments, the tapered end portion of the microfluidic body layer includes a tapered end formed along a substantially flat truncated portion of the tapered end portion.

Also described herein are microfluidic chips for electrospray ionization comprising a channel plate formed with a separation channel and at least two side channels that are each in fluid communication with at least one fluid reservoir included within the channel plate, and herein at least one side channel includes a charged coating; and a covering plate for substantially enclosing the non-intersecting fluid channels formed on the channel plate, wherein the covering plate includes an overhang that extends beyond the channel plate to provide an electrospray tip that includes an open-tip region at which each of the non-intersecting fluid channels terminate. In further embodiments, such a microfluidic chip further comprises a syringe in fluid communication with a side channel to provide sheath flow. In further embodiments, the charged coating of the side channel includes positively or negatively charged molecules. In further embodiments, the charged coating of the side channel includes negatively charged molecules, and wherein the separation channel has a charged coating that includes positively charged molecules. In further embodiments, the charged coating of the side channel is a positively charged coating, and the separation channel is without a coating. In further embodiments, the charged coating of the side channel is a positively charged coating, and the separation channel includes a neutral uncharged coating. In further embodiments, the charged coating of the side channel is a positively charged coating, and the separation channel includes a positively charged coating. In further embodiments, the charged coating of the side channel is a negatively charged coating, and the separation channel includes a negatively charged coating. In further embodiments, the coating of the side channel is a neutral uncharged coating, and the separation channel includes a neutral uncharged coating. In further embodiments, the side channel and the separation channel are uncoated. In further embodiments, the charged coating of the side channel is a negatively charged coating, and the separation channel includes a positively charged coating. In further embodiments, the charged coating of the side channel is a neutral uncharged coating, and the separation channel includes a negatively charged coating. In still further embodiments, the side channel is uncoated, and the separation channel includes a negatively charged coating.

Also described herein are any of the aforementioned microfluidic chips in which the is fabricated by pressure molding poly(styrene-co-maleic anhydride).

### Incorporation by Reference

All publications, patents and patent applications mentioned in this specification are herein incorporated by reference in their entirety to the same extent as if each individual publication, patent or patent application was specifically and individually indicated to be incorporated by reference.

### BRIEF DESCRIPTION OF THE FIGURES

A better understanding of the features and advantages of the present methods and compositions may be obtained by reference to the following detailed description that sets forth illustrative embodiments, in which the principles of our methods, compositions, devices and apparatuses are utilized, and the accompanying drawings of which:

FIG. 1 is a flowchart presenting an illustrative synthesis and use of the coated surfaces.

FIG. 2 depicts various coating embodiments which utilize amphiphilic and charged polymers.

FIG. 3 depicts various coating embodiments which utilize polymerization of hydrophobic and charged polymers.

FIG. 4A depicts various coating embodiments which utilize entrapment of neutral polymers.

FIG. 4B depicts various coating embodiments which utilize covalent attachment of charged or neutral polymers.

FIG. 5 is an illustrative schematic displaying a hydrophobic surface (a) before coating, (b) after coating with an amphiphilic polymer (PSMA), and (c) after coating the PSMA region with a charged polymer (PDADMAC).

FIG. 6 is an illustrative schematic displaying a hydrophobic surface (a) before coating, (b) after coating with an amphiphilic polymer, precursor, or monomer and (c) after coating the amphiphilic region with a charged polymer, precursor, or monomer.

FIG. 7A is an illustrative schematic displaying a hydrophobic surface coated with (a) functionalized PSMA, and (b) functionalized positively charged polymer (PCPMEDMAC).

FIG. 7B are illustrative reaction schemes for other methods to functionalize anhydride based copolymers.

FIG. 8 is an illustrative schematic displaying a hydrophobic surface (a) before coating, (b) after coating with a polymerizable hydrophobic monomers (1,14-tetradecanediol dimethacrylate), n=14, and (c) after co-polymerization of hydrophobic 1,14-tetradecanediol dimethacrylate monomers. with charged reactive monomers (3-methylammonium propylmethacrylate (MAPTAC)).

FIG. 9 is an illustrative plot of fluorescence intensity vs. time for a mixture of bodipy labeled proteins/peptides separated using an electrophoresis microfluidic chip with the separation channel coated with a 1,14-tetradecanediol dimethacrylate/MAPTAC coating.

FIG. 10A is an illustrative example of covalent attachment of a cationic polymer to a polycarbonate surface.

FIG. 10B is an illustrative example of covalent attachment of a neutral polymer to a polycarbonate surface.

FIG. 11 is an illustrative plot of fluorescence intensity vs. time for a mixture of bodipy labeled proteins/peptides separated using an electrophoresis microfluidic chip with the separation channel coated via direct covalent attachment of a cationic polymer to polycarbonate.

FIG. 12 is an illustrative schematic of a neutral hydrophilic polymer coating on and/or in a hydrophobic surface.

FIG. 13 is an illustrative schematic of a neutral hydrophilic polymer coating on and/or in a hydrophobic surface.

FIG. 14 is an illustrative schematic of a hydrophilic polymer coating that is partially entrapped in a hydrophobic surface

FIG. 15 is an enlarged perspective view of an illustrative microfluidic chip that is formed with a tip and a pair of fluid channels converging at a distal tip region.

FIG. 16A illustrates a configuration or set-up that may be incorporated with microfluidic devices including those provided elsewhere herein to provide more reliable separation and electrospray.

FIG. 16B illustrates the distal end of a microfluidic chip wherein the separation channel is coated and the side channel is coated or uncoated.

FIG. 16C illustrates the distal end of a microfluidic chip wherein the separation channel is neutrally coated or uncoated and the side channel is coated with a charged polymer.

FIG. 17 illustrates the distal end of a microfluidic chip employing two side channels for sheath flow.

FIG. 18 illustrates a multi-channel chip with sheath flow from one side and an integrated electrode positioned at the tip (3′).

FIG. 19 is a fluorescence image of a separation channel coated with PSMA-Bodipy/PDADMAC and an uncoated side channel.

FIG. 20 is a fluorescence image of separation channel coated with PSMA/MAPTAC-Bodipy and an uncoated side channel.

FIG. 21 is an illustrative plot of Mass Spectrometric detection vs. time for a mixture of native (unlabeled) proteins/peptides separated using an electrophoresis/electro-spray microfluidic chip with the separation channel coated with PSMA/PDADMAC and the side channel uncoated.

FIG. 22 presents illustrative stability data of the migration time for Bodipy-labeled ubiquitin and Angiotensin I plotted as a function of storage time.

FIG. 23 presents illustrative stability data of the theoretical plate number for Bodipy-labeled ubiquitin and Angiotensin I plotted as a function of storage time.

### DETAILED DESCRIPTION OF THE INVENTION

Methods for stably modifying a surface are needed in many different applications, including applications in the medical, biotechnology, pharmaceutical and other life sciences industries. Typically, applications in these industries utilize apparatuses/devices manufactured/fabricated from a polymer, glass, silicon, metal, or other inorganic or organic material. However, the initial surfaces of these apparatuses/devices may not have properties that are desired for a particular end user. For example, if the initial surface is hydrophobic and the end user needs a hydrophilic, positively-charged surface or region, then the original surface must be modified. Preferably, such modifications should be stable for the desired use, and even more preferably, such modifications should be stable for multiple uses. Furthermore, if such modifications are to be incorporated into a device or apparatus, then such modifications are preferably amenable to efficient, cost-effective and reproducible production. As used herein, coating refers to any means of modifying at least part of an exposed surface with another material in the form of a new region and/or layer. As described herein, the interactions between the original surface and the new region and/or layer can include hydrophobic interactions, covalent interactions, electrostatic interactions, hydrogen-bond interactions, non-covalent interactions as well as any combination of these interactions. As a result of such a coating, the properties of the new surface differ from the properties of the original surface.

One particular end use for a modified surface or region is in the field of micro-applications, including, by way of example only, miniaturized biosensors, microfluidic devices, microarrays, lab-on-a-chip devices, and other devices created on a “chip” or other miniature surface. These microfluidic devices incorporating modified surfaces or regions may be used in a variety of applications, including, e.g., the performance of high throughput screening assays in drug discovery, immunoassays, diagnostics, genetic analysis, and the like. Furthermore, these microfluidic devices incorporating modified surfaces or regions may also be used for the analysis of biological samples; wherein the biological samples may comprise, by way of example only, proteins, peptides, amino acids, steroids, fatty acids, lipids, saccharides, polysaccharides, nucleosides, nucleotides, oligonucleotides, DNA, RNA, hormones, drugs, pro-drugs, or drug metabolites.

One common surface or region that is created during the fabrication of such devices is a hydrophobic surface, whereas the final end product may have need for a hydrophilic and/or ionic surface or region. As a result, such hydrophilic and/or ionic surfaces or regions need to be created on or adjacent to the hydrophobic surface. Furthermore, for certain applications it may be desirable to control and/or tailor the surface charge density of an ionic surface. One illustrative application in which such control and/or tailoring is expected to find use is in miniaturized electrophoresis devices, i.e., allowing the fabricator to control the magnitude and direction of electroosmotic flow to suit the needs of the end user; in one example, the magnitude (regardless of sign) of the electroosmotic flow is at least 3×10⁻⁴ (cm²/vs) in a solution of 20% isopropanol and 0.05% formic acid in water.

However, because the initial hydrophobic surface and the desired hydrophilic and/or ionic surface or regions have a transition in properties, the interface is potentially unstable; thus methods for stabilizing the interface between a hydrophobic surface or region and an adjacent hydrophilic and/or ionic surface or region are in demand.

Covalent modification of a hydrophobic surface to create a hydrophilic surface is often impracticable. For certain types of hydrophobic surfaces, such as PMMA, covalent modification is limited by the functionality present on the surface, available chemistries used for attachment, and solvent systems used to enable covalent attachment to the hydrophobic surface. Often conditions must be utilized that are detrimental to the polymer, for example, the use of severe solvents and reagents, which becomes impractical for large scale manufacturing (see, e.g., S. A. Soper et al, Analytica Chimica Acta, 470, (2002), 87-99). The methodology described herein allows for modification of any hydrophobic surface, including hydrophobic surfaces that would otherwise require severe conditions in order to effect covalent modification, using solution chemistry (including, but not limited to aqueous-based methods), in a simple approach with a small number of manipulations.

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 20 carbon atoms (whenever it appears herein, a numerical range such as “1 to 10” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group could also be a “lower alkyl” having 1 to 8 carbon atoms. The alkyl group of the compounds described herein also may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkylamine” refers to the —N(alkyl)ₓH_{y} group, where x and y are selected from the group x=1, y-1 and x=2, y=0. When x=2, the alkyl groups, taken together, can optionally form a cyclic ring system.

The term “alkenyl” refers to a type of alkyl group in which the first two atoms of the alkyl group form a double bond that is not part of an aromatic group. That is, an alkenyl group begins with the atoms —C(R)═C—R, wherein R refers to the remaining portions of the alkenyl group, which may be the same or different. Non-limiting examples of an alkenyl group include —CH═CH, —C(CH₃)═CH, —CH═CCH₃ and —C(CH₃)═CCH₃. The alkenyl moiety may be branched, straight chain, or cyclic (in which case, it would also be known as a “cycloalkenyl” group).

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “amphiphilic” refers to a molecule, polymer, composition or structure that has a attraction towards both polar solvents (like a hydrophile) and non-polar solvents (like a hydrophobe). The hydrophilic portion may be neutral, positively charged or negatively charged. By way of example only, an amphiphilic polymer has hydrophobic subunits and hydrophilic subunits. Such different subunits may result from the copolymerization of more than one polymerizable molecule, at least one of which has a hydrophobic portion and one of which has a hydrophilic portion. Alternatively, an amphiphilic polymer may result from the polymerization of an amphiphilic polymerizable molecule, the co-polymerization of an amphiphilic polymerizable molecule and a non-amphiphilic polymerizable molecule, or the co-polymerization of two different amphiphilic polymerizable molecules. In yet still another variation, a hydrophobic polymer may be converted into an amphiphilic polymer by reaction with a hydrophilic reagent; the reverse situation is also envisioned, that is, a hydrophilic polymer may be converted into an amphiphilic polymer by reaction with a hydrophobic reagent.

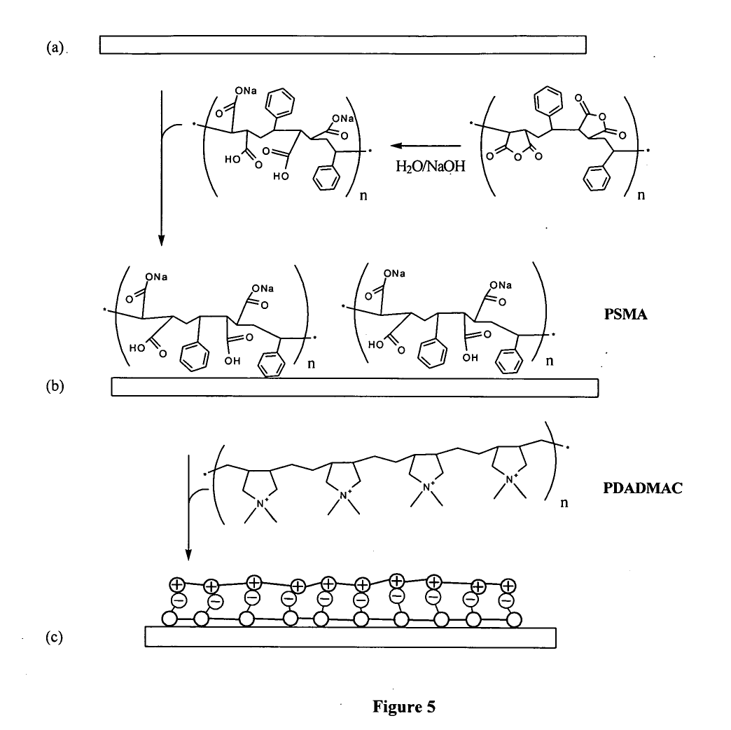

Preferably, an amphiphilic polymer should be able to coat at least a portion of a hydrophobic surface so that the predominant interactions with such a surface are through the hydrophobic portions of the amphiphilic polymer. Further, the resulting exposed surface of the amphiphilic polymer should preferably be predominantly hydrophilic. By way of example only, FIG. 5(b) presents an idealized coating of an amphiphilic polymer on a hydrophobic surface. In this figure, the amphiphilic polymer interacts with the hydrophobic surface via the hydrophobic units of the amphiphilic polymer, whereas the hydrophilic portion (here, the negatively charged units) of the amphiphilic polymer are exposed for subsequent interaction with other reagents, such as a positively-charged polymer (see FIG. 5(c)).

Many types of amphiphilic polymers and co-polymers can be designed so as to satisfy the aforementioned requirements, i.e., being able to coat a surface predominantly with one type of group while exposing to the environment a different type of group. A preferred type of co-polymer is an alternating or alt co-polymer; however, deviations from this structure are also expected to be satisfactory.

The term “aromatic” or “aryl” refers to an aromatic group which has at least one ring having a conjugated pi electron system and includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

The term “attached” refers to interactions including, but not limited to, covalent bonding, ionic bonding, electrostatic, physisorption (also referred to as physical adsorption), intercalation, entanglement, and combinations thereof.

The term “bilayer” refers to two single thin film monolayers, each of which has an average thickness less than about 500 nm. That is, each monolayer may be of a different thickness and each monolayer may also be less than 100 nm in thickness, less than 50 nm in thickness, less than 20 nm in thickness, or less than 10 nm in thickness.

The term “bond” or “single bond” refers to a chemical bond between two atoms, or two moieties when the atoms joined by the bond are considered to be part of larger substructure.

The term “coverplate” refers to a substrate used in creating certain microfluidic devices. Typically the channel network is fabricated into a separate substrate, and the separate substrate is mated or joined, at least in part, to a top substrate, forming the microfluidic device of the invention, e.g., create the channels networks. In addition, the top substrate may include a plurality of holes or ports used for fluidic introduction and/or accessibility to the channels and/or for sample introduction.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “functionalized” refers to the covalent modification of chemical moieties on a polymer.

The term “halo” or, alternatively, “halogen” means fluoro, chloro, bromo or iodo. Preferred halo groups are fluoro, chloro and bromo.

The terms “haloalkyl,” “haloalkenyl,” “haloalkynyl” and “haloalkoxy” include alkyl, alkenyl, alkynyl and alkoxy structures, that are substituted with one or more halo groups or with combinations thereof. The terms “fluoroalkyl” and “fluoroalkoxy” include haloalkyl and haloalkoxy groups, respectively, in which the halo is fluorine.

Broadly speaking, surfaces or regions interact with water in one of two ways. If the surface or region is resistant to wetting, or not readily wet by water, the interaction is termed hydrophobic. Such surfaces or regions have a lack of affinity for water. On the other hand, if the surface or region is readily wet by, or readily absorbs, water, the interaction is termed hydrophilic. Such surfaces or regions have an affinity for water. One common technique for determining whether, and to what degree, a surface is hydrophobic or hydrophilic is by contact angle measurements. In this technique, a drop of water is deposited on a test surface and the angle of the receding and advancing edges of the droplet with the surface are measured. The term “hydrophobic” is used to describe a surface or coating which forms a contact angle of greater than 60° when a droplet of water is deposited thereon. The term “hydrophilic” is used to describe a surface or coating which forms a contact angle of less than 60° when a droplet of water is deposited thereon.

The term “linkable” refers to the ability to form an attachment to a surface or region.

The term “modified hydrophobic” refers to a hydrophobic surface that has been physically and/or chemically modified; such a modified hydrophobic surface remains hydrophobic although the level of hydrophobicity may have been altered by the physical and/or chemical modification. In addition, a modified hydrophobic surface includes a hydrophilic surface that has been physically and/or chemically modified to become a hydrophobic surface.

The term “moiety” refers to a specific segment or functional group of a molecule. Chemical moieties are often recognized chemical entities embedded in or appended to a molecule.

The term “monolayer” refers to a single thin film layer that has an average thickness less than about 500 nm. That is, the monolayer may also be less than 100 nm in thickness, less than 50 nm in thickness, less than 20 nm in thickness, or less than 10 nm in thickness.

The term “multilayer” refers to multiple single thin film monolayers, each of which has an average thickness less than about 500 nm. That is, each monolayer may be of different thicknesses, and further each monolayer may also be less than 100 nm in thickness, less than 50 nm in thickness, less than 20 nm in thickness, or less than 10 nm in thickness.

The terms “nucleophile” and “electrophile” as used herein have their usual meanings familiar to synthetic and/or physical organic chemistry. Selected examples of covalent linkages formed by reaction of a nucleophile and an electrophile are given in the following table.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| Hydrazides | carboxylic acids | Hydrazines |
| N-acetyl-hydrazide | carboxylic acids | Hydrazides |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

The term “optionally substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. The protecting groups that may form the protective derivatives of the above substituents are known to those of skill in the art and may be found in references such as Greene and Wuts, above.

The term “polymer” refers to a molecule composed of smaller monomeric subunits covalently linked together. The term polymer encompasses the term homopolymer, which refers to a polymer made of only one type of monomer, as well as the term copolymer, which refers to a polymer made up of two or more types of monomer.

Examples of copolymers encompassed within the term “polymer,” as well as the shorthand terminology used within, are presented in the following table:

| Type | Shorthand | Example |
|---|---|---|
| Homopolymer | None | PolyA |
| Unspecified | -co- | Poly(A-co-B) |
| Statistical | -stat- | Poly(A-stat-B) |
| Random | -ran- | Poly(A-ran-B) |
| Alternating | -alt- | Poly(A-alt-B) |
| Periodic | -per- | Poly(A-per-B-per-C) |
| Network | net- net- | PolyA |
| Polymer blend | -blend- | PolyX-blend-polyY |
| Block copolymer | -block- | PolyX-block-polyY |
| Graft copolymer | -graft- | PolyX-graft-polyY |
| Interpenetrating polymer network | -ipn- | net-polyX-ipn-net-polyY |
| AB-crosslinked | -net- | PolyX-net-polyY |
| Starblock | star- | star-(polyX-block-polyY) |
| Segregated star | star- | star-(polyX; polyY) |

The term “sealing” refers to the method of applying a cover plate on top of a substrate in which channels have been formed in, thus enclosing, at least in part, the channels.

The term “swell” refers to a material exhibiting expansion when in contact with liquid in at least one direction i.e. in the x transverse direction, the y longitudinal direction or the z vertical direction or a material which swells in any combination of these directions.

The term “swelling” refers to the act of causing a material to swell.

The term “trilayer” refers to three single thin film monolayers, each of which has an average thickness less than about 500 nm. That is, each monolayer may have a different thickness and each monolayer may also be less than 100 nm in thickness, less than 50 nm in thickness, less than 20 nm in thickness, or less than 10 nm in thickness.

The compounds and polymers presented herein may possess one or more chiral centers and each center may exist in the R or S configuration. The compounds and polymers presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

### EXAMPLES OF POLYMERIC MATERIALS

Examples of hydrophobic polymers that may be used with the surfaces, regions, coatings, methods, devices and apparatuses described herein, include, by way of example only (note that the categories presented below are provided for organizational purposes only and not to imply that a particular polymer may not fall within more than one sub-category)
(a) polyolefins, including by way of example only, as polyethylene, poly(isobutene), poly(isoprene), poly(4-methyl-1-pentene), polypropylene, ethylene-propylene copolymers, ethylene-propylene-hexadiene copolymers, and ethylene-vinyl acetate copolymers;
(b) styrene polymers, including by way of example only, poly(styrene), poly(2-methylstyrene), styrene-acrylonitrile copolymers having less than about 20 mole-percent acrylonitrile, and styrene-2,2,3,3,-tetrafluoropropyl methacrylate copolymers,
(c) halogenated hydrocarbon polymers, including by way of example only, poly(chlorotrifluoroethylene), chlorotrifluoroethylene-tetrafluoroethylene copolymers, poly(hexafluoropropylene), poly(tetrafluoroethylene), tetrafluoroethylene-ethylene copolymers, poly(trifluoroethylene), poly(vinyl fluoride), and poly(vinylidene fluoride);
(d) vinyl polymers, including by way of example only, poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile);
(e) acrylic and acrylate polymers, including by way of example only, poly(n-butyl acetate), poly(ethyl acrylate), poly[(1-chlorodifluoromethyl)tetrafluoroethyl acrylate], poly[di(chlorofluoromethyl)fluoromethyl acrylate], poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly[5-(heptafluoroisopropoxy)pentyl acrylate], poly[1-(heptafluoroisopropoxy)undecyl acrylate], poly[2-(heptafluoropropoxy)ethyl acrylate], and poly(nonafluoroisobutyl acrylate);
(f) methacrylic and methacrylate polymers, including by way of example only, poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(methyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate);
(g) polyesters including by way of example only, poly(ethylene terephthalate), poly(butylene terephthalate), poly(ethylene terenaphthalate), and polycarbonate;
(h) anhydride based polymers, including by way of example only, poly(styrene-alt-maleic anhydride) (PSMAA), poly(styrene-co-maleic anhydride);
(i) polyacrylamides, including by way of example only, poly(N,N-dimethylacrylamide), polymethacrylamide;
(j) cyclo-olefin polymers including by way of example only, Zeonor™ and Topas™
(k) polysiloxanes, including by way of example only, polydimethyl siloxane (PDMS); and
(l) copolymers comprising at least two different monomeric subunits of any of the aforementioned homopolymers.

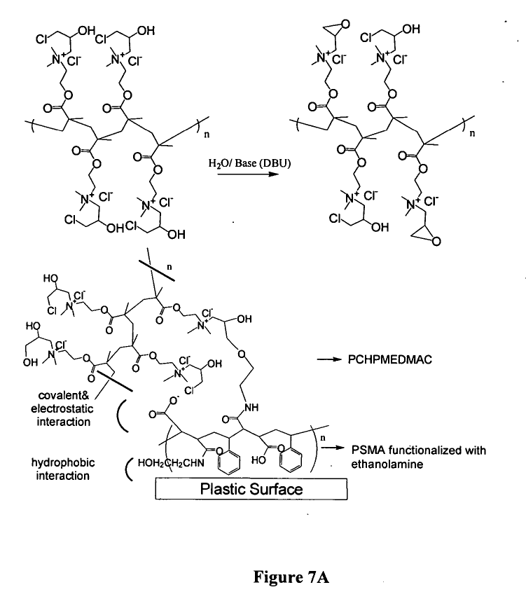

Table 2 shows examples of amphiphilic polymers that may be used with the surfaces, regions, coatings, methods, devices and apparatuses described herein, include, by way of example only (note that the categories presented below are provided for organizational purposes only and not to imply that a particular polymer may not fall within more than one sub-category). Other examples of amphiphilic polymers include, by way of example only the hydrolysis products of anhydride based polymers, such as maleic anhydride or glutaric anhydride, or polymers resulting from the reaction of anhydride polymers with nucleophiles other than water, such as those shown in FIG. 7B.

Positively charged non-amphiphilic polymers that may be used with the surfaces, regions, coatings, methods, devices and apparatuses described herein, include, by way of example only (note that the categories presented below are provided for organizational purposes only and not to imply that a particular polymer may not fall within more than one sub-category) are shown in Table 3. Alternatively, a negatively charged non-amphiphilic polymers include, by way of example only, poly(acrylic acid), poly(styrenesulfonic acid), poly(vinylphosphonic acid), poly(stryrenesulfonic acid-co-maleic acid), poly(glutamic acid), poly(aspartic acid), poly(anilinesulfonic acid), poly(3-Sulfopropyl methacrylate), polyanetholesulfonic acid sodium salt and heparin. In one embodiment, the charged non-amphiphilic polymers, used for creating the desired charge on the coated surface, possess the desired charge at or near pH 7. By way of example only, charged non-amphiphilic polymers containing amine moieties would be used to create a positively charged coating at or near pH 7; whereas, by way of example only, charged non-amphiphilic polymers containing carboxylic, sulfonic, or phosphonic acid groups would be used to create a negatively charged coating at or near pH 7.

Descriptions of Synthetic Strategies and Methodologies

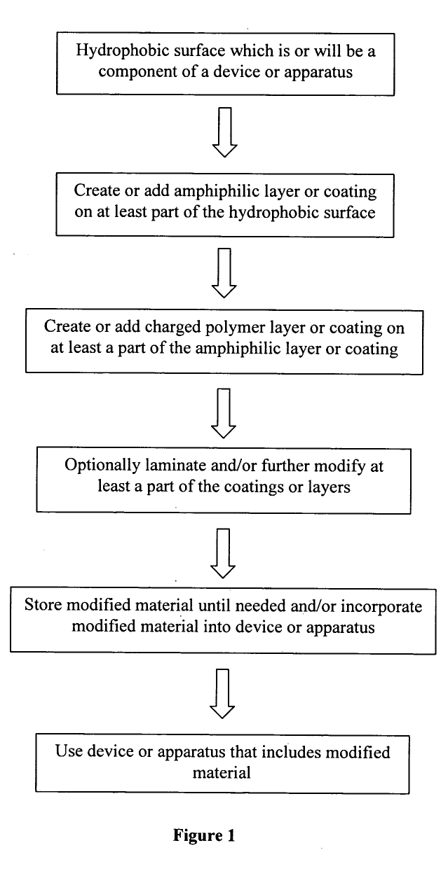

The general method for modifying a hydrophobic surface and/or region by means of an amphiphilic or modified amphiphilic polymer, as described herein, is presented in FIG. 1. The fabricator has available a hydrophobic surface and/or region which requires modification. The hydrophobic surface and/or region may be all or part of a device, apparatus, or a component of either a device or an apparatus, or the surface and/or region may become or be incorporated into a device or apparatus. Further, the hydrophobic surface may also be modified, at least in part, so that the surface region is chemically different from the non-exposed (or bulk) portion of the hydrophobic polymer. In any case, at least a part of the hydrophobic surface is coated with an amphiphilic region and/or layer. Such a coating step may occur in a single step or result from multiple sub-steps (see below). The amphiphilic coating step may occur by exposing the hydrophobic region and/or surface to an amphiphilic material (such as an amphiphilic polymer), or to a series of materials that will make an amphiphilic coating (such as an amphiphilic polymer) on the hydrophobic surface and/or region. The resulting amphiphilic region and/or layer may be a partial monolayer, a single monolayer, a partial multilayer, or it may be a multilayer, such as a bilayer; further, part of the amphiphilic region and/or layer may be embedded in the hydrophobic surface or region, or the amphiphilic region and/or layer may be a distinct surface or region adjacent to the hydrophobic region and/or layer; still further, the interaction of the amphiphilic region and/or layer with the hydrophobic surface or region may be covalent, or through non-covalent interactions, or combinations thereof. In any case, a portion of the amphiphilic region and/or layer interacts with the hydrophobic surface or region by means of the hydrophobic portion of the amphiphilic region and/or layer; at least a portion of the hydrophilic portion of the amphiphilic region and/or layer is then exposed to the environment. Further, this exposed hydrophilic portion may be ionically charged to various extents, depending upon the needs of the end user. For example, a significant ionic charge may be produced on the hydrophilic region and/or layer by reacting the hydrophilic region and/or layer with a strong acid or base; alternatively such reactions may occur prior to contacting the amphiphilic polymer with the hydrophobic surface. Further, a lesser ionic charge may be produced by reacting the amphiphilic polymer with a mixture of nucleophiles, of which only a portion comprise ionic groups.

The stability of the amphiphilic coating on the hydrophobic surface and/or region is derived in part from the hydrophobic-hydrophobic interactions between the hydrophobic surface and/or region and the hydrophobic portion of the amphiphilic coating. The thickness or properties of the amphiphilic region and/or layer need not be uniform; such non-uniformities may be a result of random fluctuations in the coating process, variations in the surface hydrophobicity, variations in buffer composition, buffer pH, flow rate, temperature, time of exposure, polymer concentration, or may result from the designs of the fabricator.

Following formation, at least in part, of the amphiphilic region and/or layer on or in (at least in part) the hydrophobic surface and/or region, the next region and/or layer may be added on or in (at least in part) the amphiphilic region and/or layer. In one embodiment, the subsequent region and/or layer is an ionically charged region and/or layer, wherein the predominant charge in the ionically charged region and/or layer is the opposite charge to the predominant ionic charge in the exposed hydrophilic surface of the amphiphilic region and/or layer. By way of example only, if the predominant charge in the exposed portion of the amphiphilic region and/or layer is a positive charge, then the predominant charge in the charged region and/or layer is preferably a negative charge; that is not to say that the only charge in the charged region and/or layer would be a negative charge, but rather that the predominant or majority charge would be a negative charge. As before, the concentration of ionic charges in the charged region and/or layer may range from a low concentration to a high concentration; further, the local charge density may vary, depending on random fluctuations of the coating process; further, the charged region and/or layer may, and most likely will, comprise non-charged moieties. If possible, an annealing step may be used to formulate a more even charge distribution within the charged region and/or layer. The charged region and/or layer need not be a charged region and/or layer upon first exposure to the amphiphilic region and/or layer; encompassed within the methods described herein, the ionic charges may be formed in the charged region and/or layer subsequent to contact with the amphiphilic region and/or layer. One of the interactions between the amphiphlic region and/or layer and the charged region and/or layer will be an ionic interaction, because as stated above, the two regions and/or layers preferably bear opposite ionic charges. However, there may also be additional interactions between the two regions and/or layers, including covalent bonds, hydrogen bonds, polar interactions, and even simple non-covalent interactions.

Although additional ionic regions and/or layers may be added on to or into the first ionically charged region and/or layer, one of the benefits of the methods, compositions and devices described herein is that this simple approach is sufficient to provide stability to the overall coating: that is, where the overall coating is comprised of a first amphiphilic region and/or layer and a second ionically charged region and/or layer. Such an approach is sufficient to provided stability even when the coating is placed on or in (at least in part) a hydrophobic surface, layer or region. For sake of simplicity, the combination of an amphiphilic region and/or layer and an ionically-charged region and/or layer will be referred to as the “two-layer coating,” although such regions and/or layers may be simple or complex and composed of a single or a multiple chemical moieties or entities, and although additional regions and/or layers may be added onto or in (at least partially) the two-layer coating.

Although not required for stability, further stability may be imparted to the coating by treating or otherwise fusing the two-layer coating. Such a treatment step may occur by means of heating, chemical reaction, ionic bombardment, γ-radiation, photochemical activation, or any other means or combination of means of treating or fusing a coating that is known in the art. In addition, such a treatment step may also occur by applying an additional region(s) and/or layer(s) onto or in (at least in part) the two-layer coating, followed (if necessary) by any of the activation methods just described. As with any of the other regions and/or layers, the treatment need not be uniform over the entire surface, not does it have to cover the entire surface. Such non-uniformity of the treated region and/or layer may result from random fluctuations of the coating process or by conscious design of the fabricator or other person(s).

The treatment step need not immediately follow the formation of the two-layer coating process; for example additional modification to the two-layer coating may occur, or additional modifications may occur on other portions of the device or apparatus of which the two-layer coating is a component, portion or feature. In addition, further modifications may occur to the two-layer coating even after the treatment step if the two-layer coating is otherwise accessible to chemical and/or biological agents, light, ions, heat, or other means of activation or modifying a two-layer coating. Examples of chemical and/or biological agents include, by way of example only, flurorophores, antibodies, peptides, ligands, catalysts, reactive groups, oligonucleotides and oligonucleosides, oligosaccharides, electron donors and electron acceptors, or a combination of such chemical agents. In addition, the treated region and/or layer may undergo further processing or modification, or the device or apparatus of which the two-layer surface is a component, portion or feature may undergo further processing, manipulation or modification until the final device or apparatus is made.

As an additional option, the unfinished or finished device or apparatus of which the two-layer coating is a component, portion or feature may be appropriately stored until further needed. Preferably, such a storage step (or even storage steps) will not result in degradation of the two-layer coating: proper storage conditions may involve control of temperature, humidity, atmosphere, or other components that may impact degradation of the two-layer coating. Further, the unfinished or finished device or apparatus of which the two-layer coating is a component, portion or feature may be stored wet, or dry.

Finally, when needed, the device or apparatus of which the two-layer coating is a component, portion or feature may be used by the end user. Examples of components, portions or features of a device or apparatus that may be coated as described herein include the separation channel of a microfluidic device, the side channel of a microfluidic device, the wells of a plate or device, sections of an array, reaction channels in a microfluidic device, storage areas on a chip or device, and the inner or outer portions of a tube. Preferably, the stability of the two-layer coating is sufficient to allow multiple uses of the device or apparatus. Furthermore, different components, features, or portions of a device or apparatus can have similar or different types of coatings, depending upon the needs of the user. The methods and coatings described herein are flexible enough to allow both the customization and the mass-production of a desired device or apparatus.

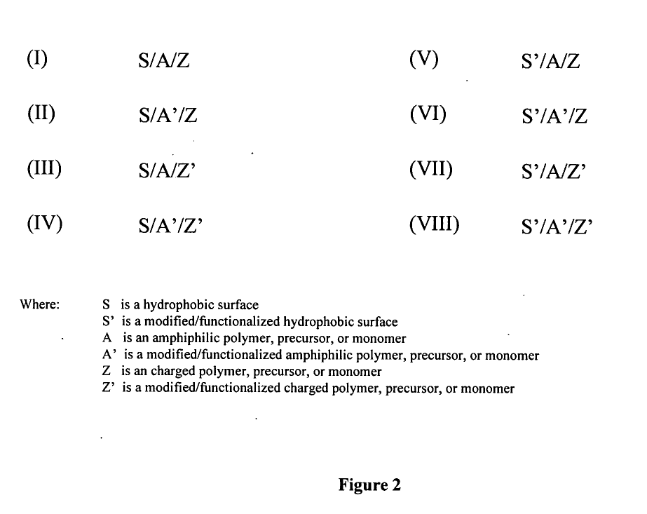

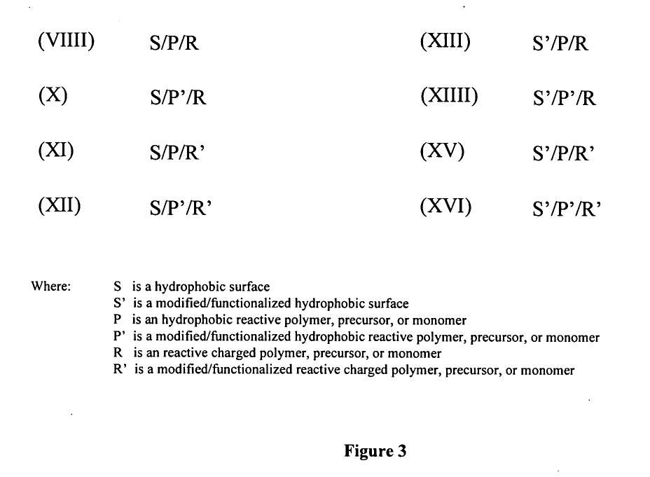

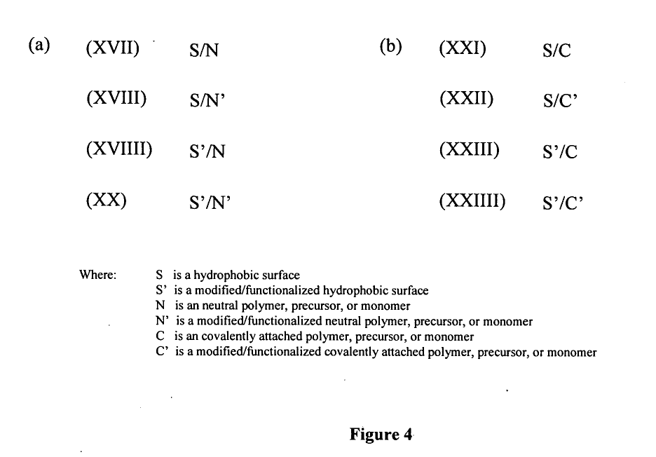

FIGS. 2-4 show various schematic embodiments of the methods and compositions described herein. FIG. 2 presents various possible configurations for at least a portion of a hydrophobic surface (any part of which may be modified, functionalized, and/or unmodified) coated with an amphiphilic polymer, precursor or monomer (any of which may be in part modified, functionalized, and/or unmodified) and with a charged polymer, precursor or monomer (any of which may be in part modified, functionalized, and/or unmodified). Various methods for achieving such coatings, as well as the characteristics of such coatings are described herein. FIG. 3 presents various possible configuration for at least a portion of a hydrophobic surface (any part of which may be modified, functionalized, and/or unmodified) coated with a reactive hydrophobic polymer, precursor or monomer (any of which may be in part modified, functionalized, and/or unmodified) and a reactive charged polymer, precursor or monomer (any of which may be in part modified, functionalized, and/or unmodified). Various methods for achieving such coatings, as well as the characteristics of such coatings are described herein. FIG. 4A presents various possible configurations for at least a portion of a hydrophobic surface (any part of which may be modified, functionalized, and/or unmodified) coated with a neutral polymer, precursor or monomer (any of which may be in part modified, functionalized, and/or unmodified). Various methods for achieving such coatings, as well as the characteristics of such coatings are described herein. FIG. 4B presents various configurations for at least a portion of a hydrophobic surface (any part of which may be modified, functionalized, and/or unmodified) coated with a covalently attached polymer, precursor or monomer (any of which may be in part modified, functionalized, and/or unmodified). Various methods for achieving such coatings, as well as the characteristics of such coatings are described herein.

In FIG. 5, presents a schematic representation in which an entire flat hydrophobic surface is coated; however, an analogous procedure may be used for any smaller portion of the surface or for any form of surface, including porous surfaces, as well as recessed, curved, twisted or other possible configurations, including the inner surface or outer surface of a tube, channel or chamber. All that is required is that chemical agents can access by some means (including pressure, percolation and diffusion) the desired surface or region. Various methods exist in the art for coating portions of a surface, including the use of masks.

The initial surface, shown at the top of FIG. 5 is a hydrophobic surface. A goal of the first step is to create an amphiphilic region and/or layer or coating on or in (at least in part) the hydrophobic surface. This coating process may (but need not) comprise multiple steps. At its most simplest embodiment, an amphiphilic polymer is applied to the hydrophobic surface. Such an amphiphilic polymer is comprised of a hydrophobic portion that forms an interaction (covalent, non-covalent, or otherwise) with the hydrophobic surface. Polar, and even ionic groups that may be components of the amphiphilic polymer may also interact with the hydrophobic surface; however, the predominant (at the least, the plurality of interactions) is an attractive interaction between the hydrophobic components of the amphiphilic polymer and the hydrophobic surface. Many methods are available for contacting the amphiphilic polymer with the hydrophobic surface, including simply exposing the hydrophobic surface to a solution containing the amphiphilic polymer, or spin coating the amphiphilic polymer onto the hydrophobic surface, chemical vapor deposition, techniques involving aerosols, and application of the pure polymer onto the surface, either as a neat solution or in vapor phase. The method of simply exposing the amphiphilic region and/or layer to a solution of the charged polymer further allows for molecular organization of the charged polymer as in interacts with the underlying amphiphilic region and/or layer. Furthermore, these aforementioned deposition methods can be undertaken at room temperature, or elevated temperature. An additional rinsing step may be utilized to remove excess amphiphilic polymer or other materials. A drying step (effected by heat, vacuum or use of drying agents) may also be included to remove excess solvent or other materials from the amphiphilic coating. The amphiphilic coating may be obtained using a) amphiphilic polymers, b) precursors to amphiphilic polymers, followed by formation of the amphiphilic polymer, or c) monomers for (a) or (b) above, followed by further reaction if needed to make the amphiphilic polymer.

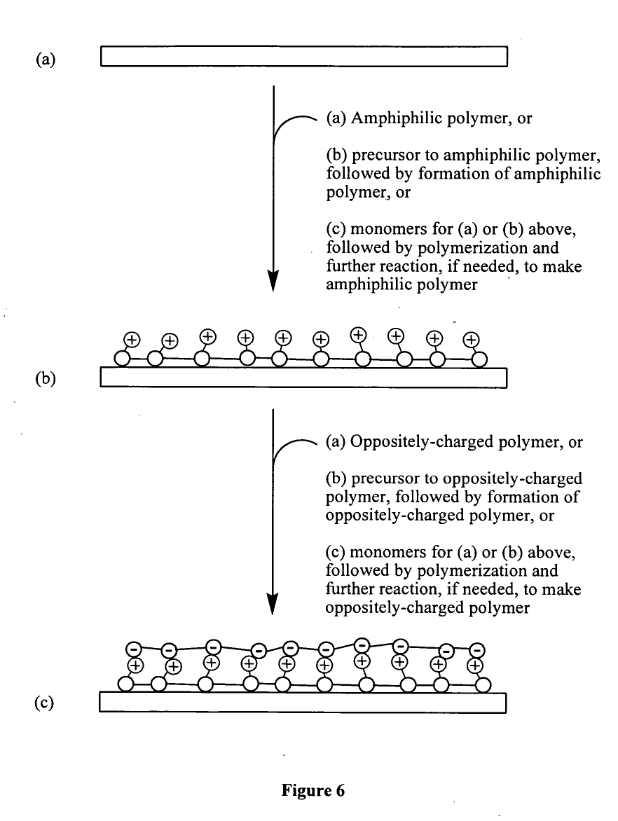

A goal of the second step in FIG. 5 is to create a charged region and/or layer or coating on or in (at least in part) the amphiphilic region and/or layer, whereby creating a stable charged “two-layer coating” on the hydrophobic surface. This coating process may (but need not) comprise multiple steps. In one embodiment, a polymer of opposite charge to that of the amphiphilic region and/or layer is applied to the amphiphilic region and/or layer on the hydrophobic surface. In the example shown in FIG. 5 the amphiphilic region and/or layer contains negatively charged moieties, while the charged polymer contains positively charged moieties, thus creating a positively charged bilayer. However, as shown in FIG. 6, a negatively charged bilayer could be formed using an amphiphilic region and/or layer containing positively charged moieties, with the charged polymer containing negatively charged moieties. Such charged polymers are comprised of charged moieties that ionically interact with the amphiphilic region and/or layer. Hydrophobic components of the charged polymer may also interact with the amphiphilic region and/or layer; however, the predominant (at the least, the plurality of interactions) is an attractive interaction between the oppositely charged moieties of the amphiphilic polymer and the charged polymer. Many methods are available for contacting the amphiphilic region and/or layer with the charged polymer, including by way of example only, exposing the amphiphilic region and/or layer to a solution of the charged polymer, or spin coating the charged polymer onto the amphiphilic region and/or layer, chemical vapor deposition, techniques involving aerosols, and application of the pure charged polymer onto the amphiphilic region and/or layer. The method of simply exposing the amphiphilic region and/or layer to a solution of the charged polymer further allows for molecular organization of the charged polymer as in interacts with the underlying amphiphilic region and/or layer. Furthermore, these aforementioned deposition methods can be undertaken at room temperature, or elevated temperature. The charged coating may be obtained using a) charged polymers, b) precursors to charged polymers, followed by formation of the charged polymer, or c) monomers for (a) or (b) above, followed by further reaction if needed to make the charged polymer. An additional rinsing step may be utilized to remove excess charged polymer or other materials. A drying step (via heat, vacuum or use of drying agents) may also be included to remove excess solvent or other materials from the coating.

In FIG. 5 is shown one embodiment of the method described herein in which the amphiphilic polymer is poly(styrene-alt-maleic acid) (PSMA) generated by base hydrolysis of poly(styrene-alt-maleic anhydride) (PSMAA) and purified prior to application onto the hydrophobic surface. The hydrophobic surface is exposed to a solution containing the amphiphilic polymer, PSMA, which adsorbs to the hydrophobic surface creating the initial amphiphilic region and/or layer. Subsequently, the PSMA region and/or layer is exposed to a solution containing the charged polymer poly(diallyldimethylammonium chloride) (PDADMAC), which ionically interacts with the amphiphilic region and/or layer creating the charged second region and/or layer on the hydrophobic surface. In this case the use of the methodology described above has modified the hydrophobic surface into a positively charged surface.

A further methodology, which incorporates the adsorption of modified amphiphilic polymers onto a hydrophobic surface, can also be used to create a positively charged, negatively charged, or neutral coating on the hydrophobic surface. Modification of amphiphilic polymers incorporates functionality into the amphiphilic polymer which can be used for subsequent attachment of a second polymer region and/or layer, thereby generating a neutral or charged region and/or layer on the modified amphilic region and/or layer. Attachment of the second polymer layer can be via electrostatic interaction or covalent linkage.

FIG. 7A shows one possible approach to the method just described. In this example the amphiphilic polymer, poly(styrene-alt-maleic acid) (PSMA), is modified by reaction with 2-aminoethanol, and a hydrophobic surface is exposed to the modified amphiphilic polymer. With aqueous chemistry a coating containing amine functionality may be created on a hydrophobic surface. This modified amphiphilic layer may then be exposed to a cationic polymer, such as poly(3-chloro-2-hydroxypropyl-2-methacryloxyethyl-dimethylammonium chloride, (PCHPMEDMAC), which has been activated by base treatment to functionalize the cationic polymer with epoxide moieties. The modified PCHPMEDMAC can electrostatically interact with the modified amphiphilic layer, and/or form covalent linkages.

Other functional groups may be incorporated into the PSMA polymer by reacting PSMAA with other nucleophiles. The use of a nucleophile, such as an alcohol, in the PSMA layer allows covalent crosslinking with cationic polymers that contain an electrophilic group, such as chlorohydrin. Additional covalent linkages may also be formed by methods known in the art; by way of example only, see the table of nucleophiles and electrophiles and the resulting covalent linkage presented above. Thus, the presence of electrophilic groups such as epoxides or chlorohydrins in the PSMA layer allows for covalent crosslinking of cationic polymers that contain nucleophiles such as alcohols or primary amino groups. Also, activation of the carboxylic acid groups of PSMA with a reagent like N-(3-dimethylaminopropyl)-N′-ethyl-carbodimide (EDC) allows the activated PSMA to be covalently crosslinked with nucleophiles such as amines or alcohols. FIG. 7B presents examples of nucleophiles that have been incorporated into maleic anhydride polymers that may be used with such covalent attachment strategies.

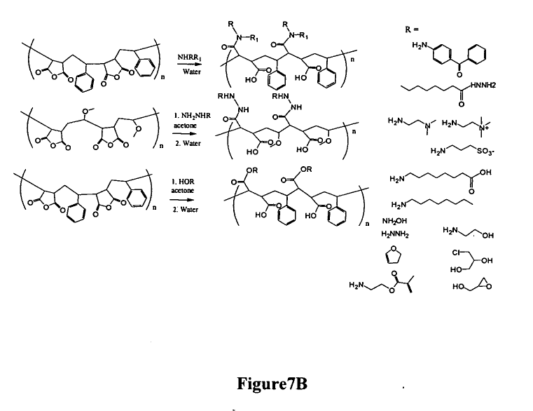

Another method for producing a very stable positively charged, negatively charged, or neutral, coating on/into a hydrophobic surface, or at least part of a hydrophobic surface, uses a radical polymerization procedure. This procedure is similar to that described in FIG. 1, however, rather than initially exposing the hydrophobic surface to an amphiphilic polymer, the hydrophobic surface is initially exposed to a polymerizable material which adsorbs on/into the hydrophobic surface. This polymerizable material contains hydrophobic regions, for interaction with the hydrophobic surface, and reactive moieties to accomplish covalent linkage (including co-polymerization) with neutral or charged reactive monomers, thus producing in effect an amphiphilic polymer. A possible embodiment of the method and compositions described herein is presented in FIG. 8 in which the polymerizable material is initially adsorbed on/in the hydrophobic surface, and a charged monomer species that subsequently reacts with the absorbed polymerizable material. In this particular example n is equal to 14, however the value for n may from 2 to 30. In FIG. 8 an entire flat surface is covered by the resulting amphiphilic polymer; however, an analogous procedure may be used for any smaller portion of the surface or for any form of surface, including porous surfaces, as well as recessed, curved, twisted or other possible configurations, including the inner surface or outer surface of a tube, channel or chamber. Chemical agents should be able to access by some means (including pressure, percolation and diffusion) the desired surface or region. Various methods exist in the art for coating portions of a surface, including the use of masks.

The initial surface, shown at the top of FIG. 8 is a hydrophobic surface. A goal of the first step is to create a reactive layer or coating on or in (at least in part) the hydrophobic surface. This coating process may (but need not) comprise multiple steps. In one embodiment, a hydrophobic polymer with reactive moieties is applied to the hydrophobic surface. Such a hydrophobic polymer is comprised of a hydrophobic portion that forms an interaction (covalent, non-covalent, or otherwise) with the hydrophobic surface. Polar, and even ionic groups that may be components of the hydrophobic polymer may also interact with the hydrophobic surface; however, the predominant (at the least, the plurality of interactions) is an attractive interaction between the hydrophobic components of the hydrophobic polymer and the hydrophobic surface. Many methods are available for contacting the hydrophobic polymer with the hydrophobic surface, including simply exposing the hydrophobic surface to a solution of the hydrophobic polymer, or spin coating the hydrophobic polymer onto the hydrophobic surface, chemical vapor deposition, techniques involving aerosols, and application of the pure polymer onto the surface. An additional rinsing step may be utilized to remove excess hydrophobic polymer or other materials. A drying step (effected by heat, vacuum or use of drying agents) may also be included to remove excess solvent or other materials from the hydrophobic coating. This results in a polymeric coating on/in the hydrophobic surface which has pendent reactive moieties, such reactive vinyl groups, used for subsequent radical polymerization with a charged species.

A goal of the second step in FIG. 8 is to create a charged layer or coating on or in (at least in part) the polymeric layer, whereby creating a stable charged bilayer on the hydrophobic surface. This coating process may (but need not) comprise multiple steps. In one embodiment, a charged monomer, or a charged polymer with reactive moieties is applied to the absorbed polymeric layer on the hydrophobic surface followed by subsequent free-radical polymerization. Initiation of the free-radical polymerization process may be accomplished using heat, exposure to UV, and any other method known in the art. In the example shown in FIG. 8, 3-methylammonium propylmethacrylate (MAPTAC) is co-polymerized via free-radical polymerization to create a positively charged layer covalently attached to the hydrophobic layer adsorbed on/in the hydrophobic surface. Although, the example demonstrates formation of a positively charged bilayer, the same methodology can be used to create a negatively charged bilayer. An additional rinsing step may be utilized to remove excess materials not bound to the adsorbed polymeric layer on/in the hydrophobic surface. A drying step (effected by heat, vacuum or use of drying agents) may also be included to remove excess solvent or other materials from the bilayer coating.

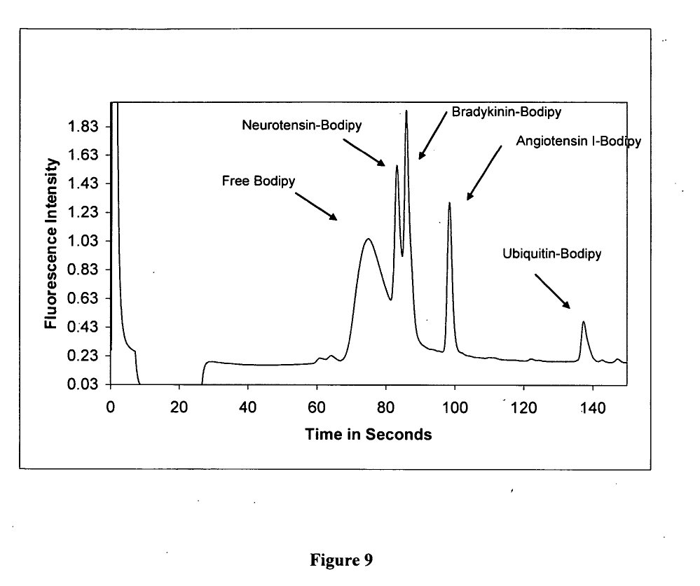

The methods described above create a bilayer to modify the surface characteristics of a hydrophobic surface. However, the hydrophobic surface can also be modified by covalent attachment of positively charged, negatively charge, or neutral polymers to generate positively charged, negatively charge, or neutral layers, respectively, on the hydrophobic surface. In the case of polycarbonate, the phenolic functionality of the surface can be used for reaction with chlorohydrin modified polymers, thus creating any desired surface characteristic from a wide range of chlorhydrin modifiable polymers; either positively charged, negatively charged or neutral. FIG. 10A-10B depict examples of this approach, in particular FIG. 10A shows the covalent attachment of poly(3-chloro-2-hydroxypropyl-2-methacryloxyethyldimethylammonium chloride) onto polycarbonate, while FIG. 10B shows covalent attachment of polyethylene oxide derivatives to polycarbonate. Alternatively, chemistry can be performed on the residual chlorohydrin groups.

Yet another embodiment utilizing covalent attachment of neutral hydrophilic polymers to hydrophobic surfaces is, by way of example only, reacting poly(ethylene glycol-co-maleic anhydride) (PEG-AO-Mal) with a surface with available nucleophiles. Also, any amino reactive polyethylene glycol molecule could be used in a similar manner. This modification imparts a neutral hydrophilic coating on the hydrophobic surface, which yields minimal or no EOF. This modified surface is also useful for resisting adsorption of protein from solution.

Another example of direct covalent attachment to the hydrophobic surface is to react polycarbonate with copolymers containing oligo ethylene glycol groups and chlorohydrins.

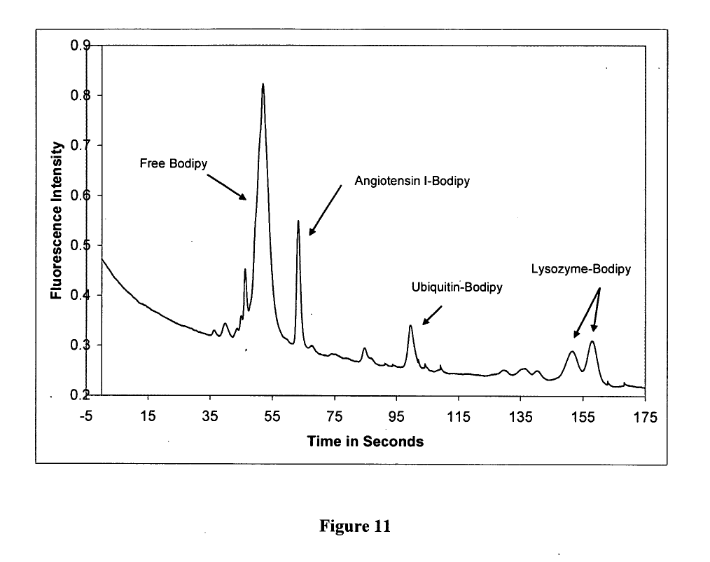

Another embodiment involves exposing hydrophobic surface to PSMA which has been functionalized with electrophilic groups. This modified surface is then reacted with polyethylene glycol bearing nucleophilic moieties, such as, by way of example only, amino-terminated polyethylene glycol, thus forming a bilayer with exposed hydrophilic moieties on the original hydrophobic surface. This embodiment is presented schematically in FIG. 12. This approach may be extend to any hydrophobic surface that can be functionalized with electrophilic groups, including, by way of example only, chlorohydrides, carboxylates, aldehydes, and or ketones.

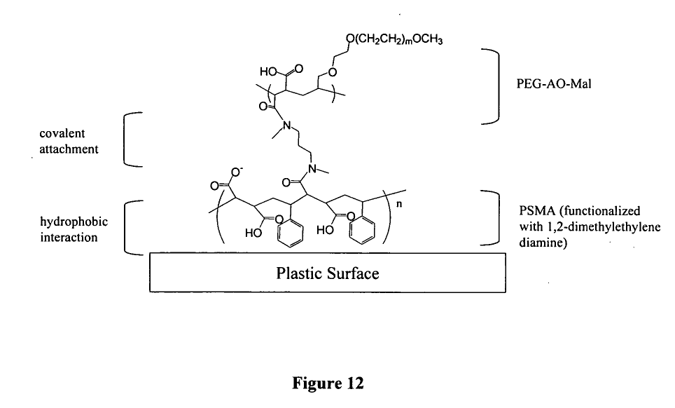

FIG. 13 shows an embodiment for the generation of a trilayer. The example shown is for a neutral coating; however this approach may also be extended to creating positively charged or negatively charged coatings. In this embodiment, PSMA is used to coat a hydrophobic surface via hydrophobic interaction, the resulting surface is then exposed to a functionalized polyionic polymer which electrostatically interacts with the PSMA surface. To complete the trilayer, the functionalized polyionic polymer is reacted with functionalized polyethylene glycol. The functional group on the polyethylene glycol polymer can be nucleophilic or electrophilic, depending on the functional groups on the polyionic polymer.

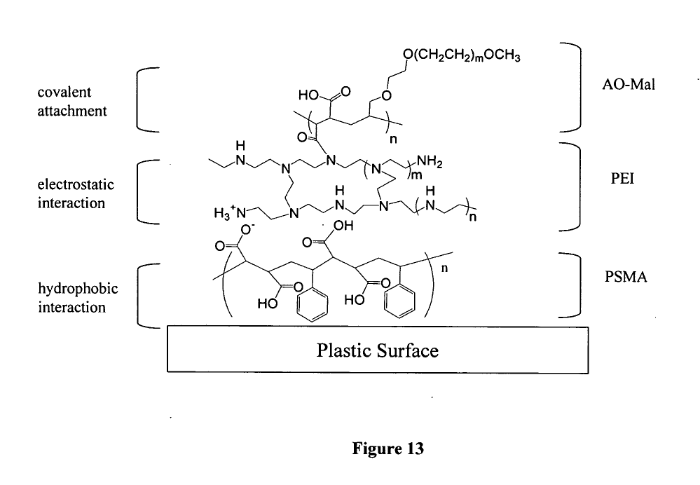

Alternatively, a simple surface modification method that can be used to modify the surface characteristics of hydrophobic surfaces involves the following procedure. For example, assuming material A has the desired characteristics and the surface of material B is to be modified to possess the property of material A. Material A is dissolved in a solvent which swells/attacks/penetrates material B and material B is then exposed to this solution. During the time of exposure, material A physically interpenetrates the surface networks of material B, becomes embedded in the surface of material B. After exposure to the material solution, material B is dried, leaving the surface blended with material A. By way of example only, the method can be used to modify the hydrophobic surfaces of poly(methyl methacrylate) (PMMA) or polycarbonate (PC) with hydrophilic polymers; poly (ethylene oxide) (PEO) or hydroxypropyl methyl cellulose (HPMC). These hydrophilic polymers are dissolved in either a solution of at least 50% isopropanol for the PMMA surface or at least 50% acetonitrile for the PC surface. The PMMA or PC surfaces are then exposed to the respective solutions and then dried. The contact angle of water on the subsequently modified surfaces is smaller than the un-treated surfaces, suggesting that the surfaces have become more hydrophilic after blending in the hydrophilic polymer. FIG. 14 shows an schematic of the entrapment of HPMC in PMMA.

The surface of anhydride based copolymers, such as, by way of example only, poly(styrene-co-maleic anhydride) (PSMAA), are reactive towards nucleophiles, such as amino groups. Additional examples of other anhydride base copolymers and nucleophiles used to modify them can be found in Table 2 and FIG. 7B, respectively. Furthermore; these copolymers can be pressure molded into any desired configuration and used as the bulk material for a component or apparatus of interest. For example, PSMAA can be pressure molded to form microfluidic channels in a microfluidic apparatus. Treatment of the PSMAA surface with a polyamine under basic conditions covalently attaches the polyamine and generates a stable, hydrophilic surface in a one step procedure. This procedure can be applied prior to/or after sealing of the molded parts to create the microfluidic channel. Sealing of the molded parts with a cover plate can be achieved using lamination, ultra-sonic welding, and thermal bonding, or any other technique known to one skilled in the art. Reaction with a polyamine generates a positive charged surface; however, reaction of the PSMAA with an amino functionalized PEG derivative can generate neutral surfaces.

Microfluidic Devices

Microfluidic chips are often constructed using conventional semiconductor processing methods including photolithographically masked wet-etching and photolithographically masked plasma-etching, or other processing techniques including embossing, molding, injection molding, photoablating, micro-machining, laser cutting, milling, and die cutting. These devices conveniently support the separation and analysis of sample sizes that are as small as a few nanoliters or less. In general, these chips are formed with a number of microchannels that are connected to a variety of reservoirs containing fluid materials. The fluid materials are driven or displaced within these microchannels throughout the chip using electrokinetic forces, pumps and/or other driving mechanisms. The microfluidic devices available today can conveniently provide mixing, separation, and analysis of fluid samples within an integrated system that is formed on a single chip.

There are numerous design alternatives to choose from when constructing an interface for microfluidic chips and electrospray ionization mass spectrometers. Some electrospray ionization interfaces include microfluidic chips that attempt to spray charged fluid droplets directly from the edge of the chip. But the accompanying solvent is known to wet much of the edge surface of the chip so as not to offer a high-stability spray for many applications. Other attempts to spray ionized particles directly from the edge of a microfluidic chip edge therefore rely on the formation of a hydrophobic surface that can yield improved spray results; however, even that often proves to be insufficiently stable. At the same time, adequate results can be also achieved with other chip devices that incorporate fused silica capillary needles or micro-machined or molded tips. In particular, some recent electrospray ionization designs incorporate small silicon etched emitters positioned on the edge of a microfluidic chip. While it is possible to generate a relatively stable ionization spray for mass spectrometric analysis with some of these microfluidic devices today, they generally require apparatus that is relatively impractical and economically unfeasible for mass production.

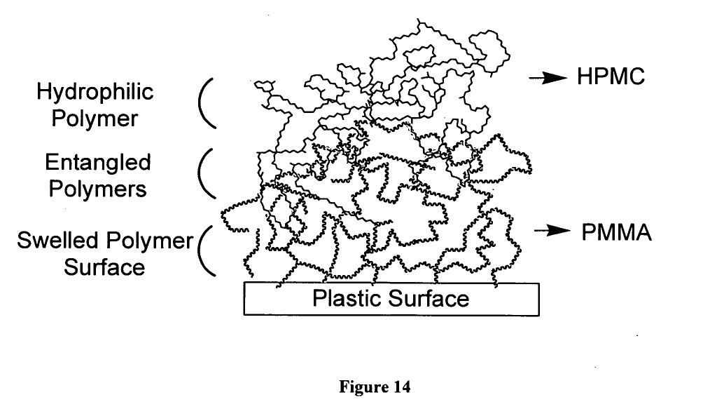

In one aspect described herein, are methods for providing coatings for multi-channel microfluidic chips and devices; examples of such chips and devices are described in U.S. patent application Ser. Nos. 10/649,350 and 10/871,498, which are herein incorporated by reference in their entirety. One embodiment provides microfluidic chips that are formed with individual fluid channels. Such fluid channels extend through the body of the microfluidic chip and converge at a common distal tip region. The distal tip region includes an open-ended distal tip formed along a defined surface of a microfluidic chip body. The microfluidic chip may be constructed from a pair of polymer plates in which the converging channels run through and lead up to the distal tip region. The microfluidic chip can be also formed with multiple but separate channels that supply fluids such as samples and sheath flow solutions to a single common electrospray tip. One method for achieving the interface between the microfluidic device and a mass spectrometer is illustrated by the three-dimensional representation in FIG. 15. In FIG. 15, a microfluidic chip 10 for electrospray ionization (ESI) applications is formed with multiple fluid channels 12 converging at a distal tip region 14. The fluid channels 12 may be formed on a substrate layer 16 of the chip 10 that is composed of glass, quartz, ceramic, silicon, silica, silicon dioxide or other suitable material such as a polymer, copolymer, elastomer or a variety of commonly used plastics. The channels 12 can be created using a variety of methods, such as conventional semiconductor processing methods including photolithographically masked wet-etching and photolithographically masked plasma-etching, or other processing techniques including embossing, molding, injection molding, photoablating, micro-machining, laser cutting, milling, and die cutting. A variety of channel patterns and configurations may be also selected for the channels, including channels having a substantially rectangular, trapezoidal, triangular, or D-shaped cross-section. For example, these channels may be produced with an anisotropically etched silicon master having a trapezoidal or triangular cross-section. A channel having a D-shaped cross-section may be formed alternatively following isotropic etching processes. The pair of channels 12 formed on the substrate layer 16 can run relatively non-parallel as shown with respect to each other which substantially converge at the distal tip region 14. A cover plate 5 can be bonded to the substrate layer 16, whereby sealing the cover plate 5 onto the substrate 16 and enclosing the channels 12. The cover plate 5 is formed so as to terminate at the end of the channels 12 at the distal tip region 14. The distal tip region 14 of the ESI tip 15 may be formed with an open-ended construction where different fluids can emerge or emit therefrom for analysis by a mass spectrometer or other analytical apparatus or detection method. In addition, the open distal tip region 14 can be created in the embossed substrate layer 16 or in the cover plate 5.

In another aspect described herein, are coating methods that may be used with multi-channel microfluidic chips and devices that additionally have features to provide improved fluid flow control, with or without using sheath flow for electrospray stability. As an additional aspect described herein are the microfluidic chips and devices that include the feature that provide improved fluid flow control, with or without using sheath flow for electrospray stability. Reliable methods and apparatus are provided for achieving stable electrospray with or without sheath flow on microfluidic chips. The microfluidic chips include (1) separation or main channels with charged coatings and side channels with charged coatings or without coatings that maintain stable separation and electrospraying; (2) separation or main channels with neutral coatings and modified side channels with charged coatings that maintain stable separation and electrospraying during application of a sheath flow as provided herein. The side channels can be used for sheath flow assisted electrospray, or sheathless electrospray. For the application of sheathless electrospray, the function of the side channel is to establish electrical contact and whereby allow for generation of an electrospray. These techniques and microfluidic devices can assist in system automation, and reduce system complexity. At the same time, the electrospray devices provided with such an embodiment can increase system reliability and allow for relatively longer separation times. The sheath flow provided by the microfluidic side channels can be driven by pressure and/or electroosmotic flow. The microfluidic chips and devices used for electrophoresis, for example, those described in U.S. patent application Ser. No. 10/649,350, can be coupled with a mass spectrometer to deliver an electrospray by either sheath flow assisted techniques or sheathless flow.

For sheathless applications, an electrospray may be achieved by conventional methods such as pressure or electroosmotic flow (EOF) in a separation channel. Meanwhile, when a sheath flow is applied as with certain applications of the invention herein, a more stable electrospray can be observed that can facilitate system optimization and calibration. In the past, sheath flow was initially used in capillary CE/MS systems and was later adopted for microchip-based CE/MS platforms such as those herein. By inserting a capillary tube to the chip to serve as an extension of the microchannel, a sheath flow interface with the capillary can be provided to assist and stabilize electrospraying from a microfluidic chip. Usually a syringe is connected to a sheath flow channel through Upchurch fitting or other acceptable fixtures, and a metal connector is placed in a fluid line positioned between a well or reservoir in a microfluidic chip and the syringe. However the following problems and other issues arise with this conventional setup which is addressed by this aspect of the invention: (1) bubbles will be often generated in the line during the electrophoresis and electrospray, and these bubbles could terminate the experiment under certain conditions such as when the applied current is >5 μA; and (2) the reliable sealing of the sheath flow loop could pose a problem and leak.

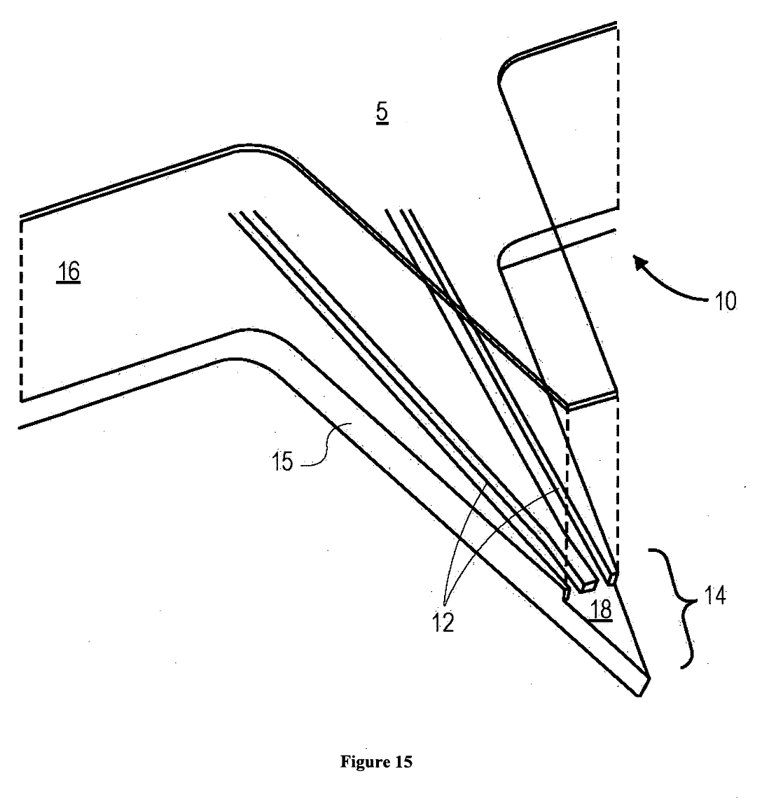

FIG. 16A illustrates a sheath flow configuration or set-up that may be incorporated with microfluidic devices including those provided elsewhere herein to provide more reliable separation and electrospray. In this configuration, four electrodes may be selected to provide fluid control within the device including a sheath flow emanating from a side channel via EOF to achieve bulk movement of aqueous solutions therein past stationary channel wall surfaces upon application of an electric field, that is upon application of current or voltage. To provide sheath flow via EOF action, an electrode is dipped in Well #3 that is in fluid communication with a side channel. FIG. 16A also illustrates a configuration or set-up for separation and sheathless electrospray from microfluidic chips. In this case, the side channel is only used for electric contact. A coating selected for the side channel can be positive, negative, neutral, or no coating based on the surface charge states in the main separation channel, or channels. As illustrated in FIG. 16B, the side channel may be coated negatively, neutrally, or no coating when a main separation channel has a positive coating (positive ion mode), or the side channel may be coated positively, neutrally, or no coating when a main separation channel has a negative coating (negative ion mode). In another preferable embodiment, as shown in FIG. 16C, the side channel may be coated positively (positive ion mode) or negatively (negative ion mode) when the main separation channel includes a neutral coating or no coating at all (non-coating). The positive, negative or neutral charge coatings herein can be formed by lining channel walls as already described above. The desired electrical parameters, such as current, voltage, or power, selected for the separation of a sample in the main channel and electrospraying at the device tip are achieved by selectively applying a combination of voltages or currents in Wells #1, #2, #3 and #4. The presence of bubbles often generated on the electrodes during the separation and electrospray will therefore not readily enter into the channels of the microfluidic chip, if at all, and will thus not affect significantly or terminate a separation process. It shall be understood that these channel configurations may be formed in the body or channel layer portions of microfluidic chips herein and combined with other systems and aspects of the invention described throughout this disclosure.

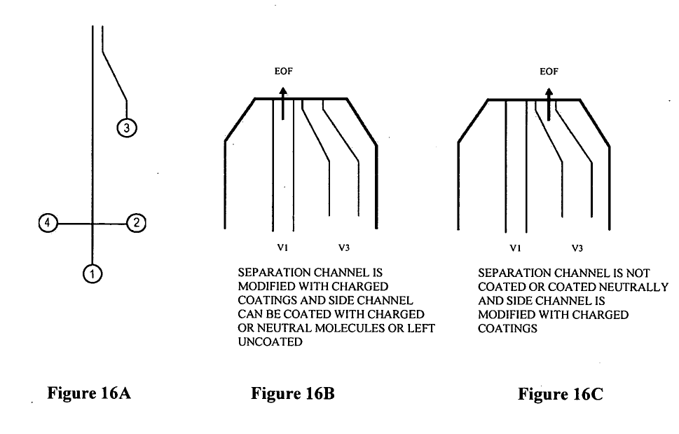

FIG. 17 shows another variation of the invention that includes a four electrode approach but with two side channels for both sheath flow and electrical contact. As shown in other portions of this specification, multi-channel microfluidic chips herein can include channel layers formed with a plurality of separation and/or side channels to support various electrospray related functions. In this illustrated embodiment, a first side channel connected to a Well #5 is used for providing the sheath flow through a syringe, and a second side channel is mainly for electrical contact by dipping an electrode in corresponding Well #3. This configuration allows the sheath flow to change flexibly and allows for system optimization more easily and more reliable electrospray. To prevent the separated charged species of the separation channel from entering into the side channel leading from Well #3 in this case as illustrated, the side channel can be coated in the same way as previously described with FIGS. 16A-C. Moreover, this separation/side channel configuration can provide a sheath flow using a syringe that is connected to Well # 5 and its respective side channel and/or via EOF in another side channel connected to Well #3 that includes the electrode dipped into therein. Alternatively, the side channel connected to Well #5 can be also coated to prevent the separated charged species from the separation channel from entering therein. These coating can be positive, negative, neutral, or no coating at all based on the surface charge states in the main separation channel as explained previously. For certain applications, the separation channel may remain uncoated or contain a neutral uncharged coating. The desired electrical parameters, such as current, voltage, or power, required for the separation in the main channel and electrospray at the tip can be also achieved by applying voltages or currents in Wells #1, #2, #3 and #4 as described previously.

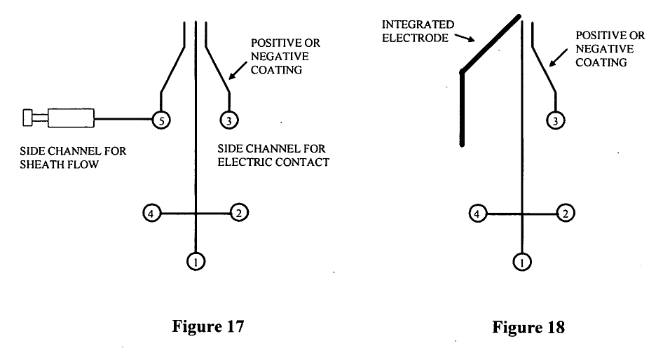

FIG. 18 describes another variation of the invention to provide a multi-channel chip with sheath flow similar to those previously described except that an integrated electrode is positioned at the tip (3′). This alternative design and method of electrospraying employs five electrodes in total and can provide direct control in the separation and electrospray electrical parameters. The task of electrospray optimization can be thus accomplished much easier with this configuration. Sheath flow can be provided by EOF in a side channel connected to Well #3 where an electrode is dipped therein. A positive or negative charged coating can be applied to the side channel walls leading from Well #3 in order to prevent charged species from entering therein. The electrical parameters, such as current, voltage, or power, required to effect separation in the main channel and electrospray at the tip can be achieved by applying voltages or currents in Wells #1, #2, #3, #4 and at electrode 3′. Methods are thus provided herein for improved fluid control in a microfluidic chip with sheath flow for enabling separation and more stable electrospray. A microfluidic chip or device may be selected as an initial step having a separation channel and at least one side channel for providing sheath flow. The side channel may include a positively or negatively charged coating with molecules having groups of suitable charges exposed to sheath flow solutions therein. A sample may be introduced into a fluid well on the chip and directed to the separation channel whereupon electrical parameters can be applied to a network of wells and channels through a series of electrodes so that selected components therein can be electrophoretically separated and emitted from the microfluidic chip as an electrospray into a mass spectrometer for analysis. The separation process and stable electrospray can be therefore achieved substantially without any of the charged species from the separation channel from entering the side channels having positively or negatively charged coatings. It shall be understood that the application of voltages or currents to create electric fields can be carried out using known microfluidic control systems.

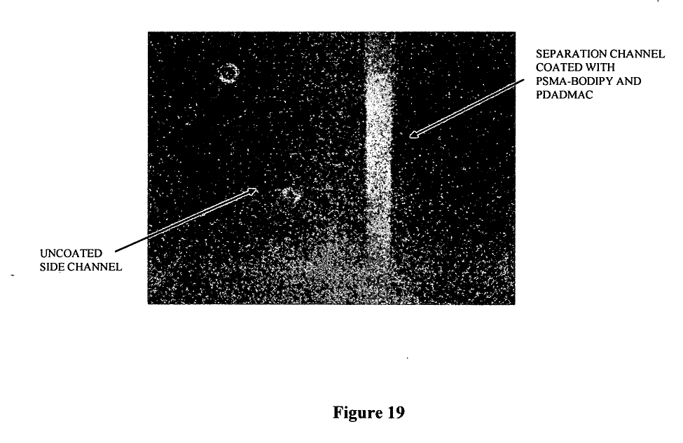

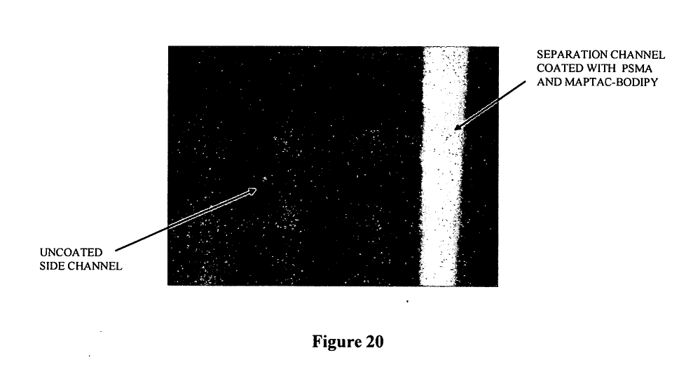

FIG. 19 is photograph illustrating the selective coating of the separation channel, relative to the side channel, in which the separation channel has been coated with PSMA labeled with bodipy and then this fluorescent coating was electrostatically coated with PDADMAC. Similarly, FIG. 20 also illustrates the selective coating of the separation channel, relative to the side channel, however, in this example the separation channel has been coated with unlabeled PSMA and this coating was electrostatically coated with bodipy labeled MAPTAC. Both images show that the separation channel is selectively coated, while the side channel remains uncoated. Although theses photographs illustrate the ability to obtain selective coatings, it may be desirable to manufacture and utilize the microfluidic devices described above with both the separation channels and side channels having a positive coating. Additionally, it may be desirable to manufacture and utilize the microfluidic devices described above with both the separation channels and side channels having a negative coating. Further, it may be desirable to manufacture and utilize the microfluidic devices described above with both the separation channels and side channels having a neutral coating. Still further, it may be desirable to manufacture and utilize the microfluidic devices described above with both the separation channels and side channels uncoated. Additionally, it may be desirable to manufacture and utilize the microfluidic devices described above with the separation channels having a negative coating and the side channels uncoated. Further, it may be desirable to manufacture and utilize the microfluidic devices described above with the separation channels having a negative coating and side channels having a neutral coating. Still further, it may be desirable to manufacture and utilize the microfluidic devices described above with the separation channels having a positive coating and the side channels having a negative coating.

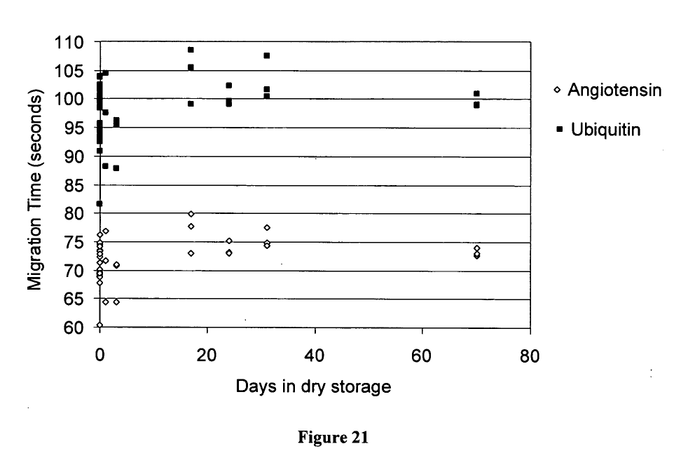

FIG. 21 shows an electropherogram of a mixture of proteins using mass spectrometric detection. The microfluidic device used for this exemplary separation utilized a separation channel selectively coated with PSMA/PDADMAC, and an uncoated side channel. In this example the side channel was used as a means to provide electrical contact to the electrospray tip.

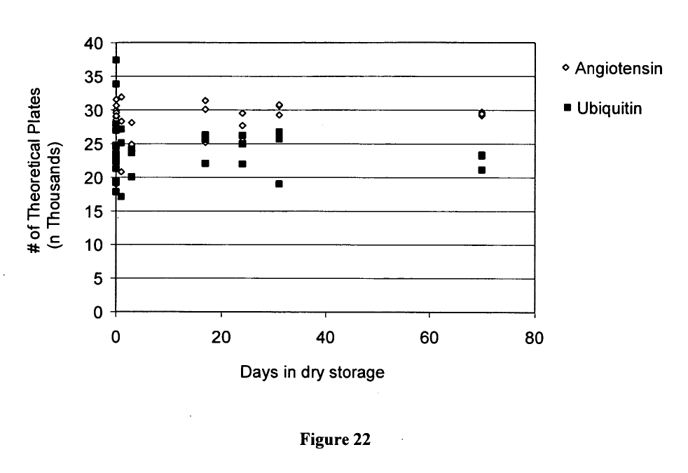

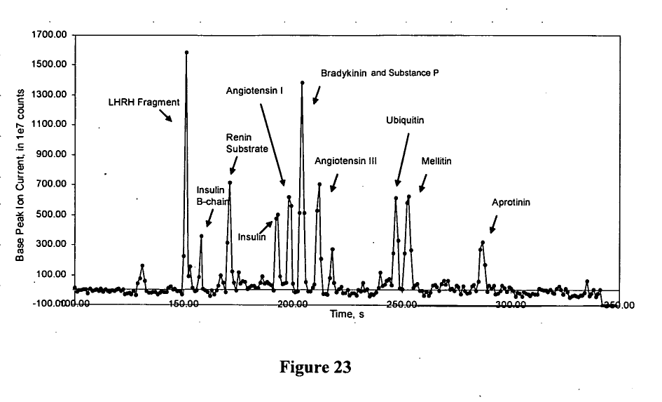

The stability of the PSMA/PDADMAC coatings is shown in FIGS. 22 and 23. In FIG. 22 the migration time of bodipy-labeled ubiquitin and bodipy labeled Angiotensin I as function of days stored is shown, while, in FIG. 23, the number of theoretical plates for bodipy-labeled ubiquitin and bodipy labeled Angiotensin I as a function of days stored is shown. See example 11 for details. The data suggests that the bilayer as produced is stable for at least 60 days.

The following examples are provided to further illustrate our devices, compositions and methods and are not provided to limit the scope of the current invention in any way.

### EXAMPLES

### Example 1

### Preparation of PSMA-PDADMAC Coating

Materials and solvents were analytical grade or better and were purchased from commercial vendors unless otherwise noted. 1,14-tetradecanediol dimethacrylate, 1,8-diazabicyclo[5.4.0]undec-7-ene (DBU), glycidol, TEMED, [3-(methacryloylamino)propyl]trimethylammonium chloride solution (MAPTAC), poly(diallyldimethylammonium chloride, poly(styrene-alt-maleic anhydride) (PSMAA), poly(styrene-co-maleic anhydride), 2,3-dihydrofuran, 2-aminoethyl methacrylate hydrochloride, poly(ethylene glycol) methyl ether methacrylate, 4-aminobenzophenone, octanohydrazide (fix in FIG. 7B), (2-aminoethyl)trimethylammonium chloride hydrochloride, 3-amino-1-propanesulfonic acid, 8-aminooctanoic acid, 1-octanamine, N,N-dimethylethylenediamine, N,N′-dimethylethylenediamine, 3-chloro-1,2-propanediol, (hydroxypropyl)methyl cellulose, branched polyethyleneimine (PEI), 4-acryloylmorpholine and ammonium persulfate as well as all peptides and proteins were purchased from Sigma/Aldrich/Fluka (also referred to herein as “Aldrich”) (Milwaukee, Wis.); HPLC grade water (Burdick and Jackson), acetone (Burdick and Jackson), isopropanol (Burdick and Jackson), and sodium hydroxide (Baker) were purchased from VWR Scientific; 2-aminoethanol was purchased from TCI America. Poly(2-hydroxy-3-methacryloxypropyl-trimethylammonium chloride), poly(3-chloro-2-hydroxypropyl-2-methacryloxyethyl-dimethylammonium chloride) (PCHPMEDMAC) and poly(ethylene glycol) methyl ether methacrylate were purchased from PolySciences Inc. Warrington, Pa. N-(4,4-difluoro-5,7-dimethyl-4-bora-3a,4a-diaza-s-indacene-3-propionyl)cysteic acid, succinimidyl ester, triethylammonium salt
(BODIPY® FL, CASE, cat.#D6141) and 4-difluoro-5,7-dimethyl-4-bora-3a,4a-diaza-s-indacene-3-propionyl ethylenediamine, hydrochloride

(BODIPY® FL EDA cat.#D2390) were purchased from Molecular Probes, Eugene, Oreg. AO-MAL was purchased from Shearwater polymers, now Nektar Therapeutics.

TABLE 1
Name, structure and potential source for various reagents used in Example 1.

| Name | Chemical Structure | Source |
|---|---|---|
| Poly(styrene-alt- maleic anhydride) (PSMAA) | 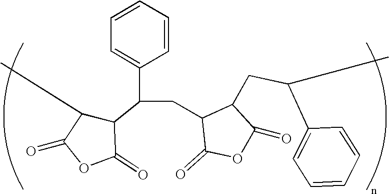 | (MW ˜350,000), available from Aldrich as the partial methyl ester. |
| poly(styrene-alt- maleic acid) (PSMA) | 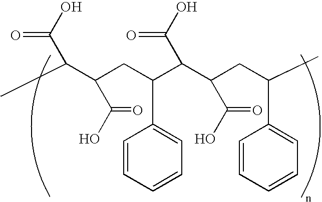 | Made via hydrolysis of PSMAA or available from Aldrich (Mw ˜120,000). |
| poly(diallyl- dimetylammonium chloride)¹(PDADMAC) | 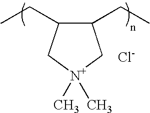 | Available from Aldrich (Mw ˜400,000-500,000) as a 20% solution in water. |

¹PDADMAC is available from Aldrich as a 20% w/v solution in water in low, medium or high molecular weights (100,000-200,000; 200,000-350,000; and 400,000-500,000, respectively).

### Example 1A

### Synthesis of poly(styrene-alt-maleic acid) (SMA) from poly(styrene-alt-maleic anhydride) (SMAA).

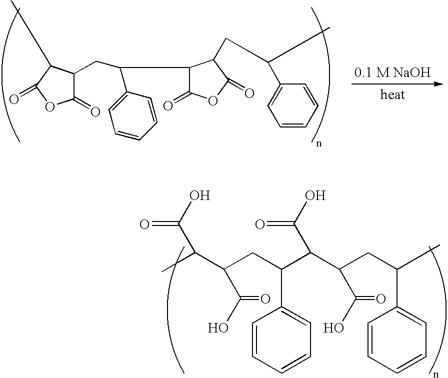 ¹ PDADMAC is available from Aldrich as a 20% w/v solution in water in low, medium or high molecular weights (100,000-200,000; 200,000-350,000; and 400,000-500,000, respectively).

A 10% w/v solution of poly(styrene-alt-maleic anhydride) (PSMAA, M_{W} 350,000) was prepared by dissolving 2.0 g of PSMAA in 20 mL of acetone. To this solution, 1 mL of water was added with vigorous mixing and the resulting solution stirred overnight. The partially hydrolyzed PSMAA acetone solution was added dropwise to a rapidly stirred aqueous solution of sodium hydroxide at 80-90° C. (0.1 M, 180 mL). The solution was cooled and the pH was adjusted to ˜6 using hydrochloric acid (6.0 M). Water was added to give a total volume of 200 mL resulting in a 1% w/v solution of PSMA. A commercial PSMA polymer is also available.

### Example 1B

### Preparation of a Bilayer Coating of PSMA-PDADMAC:

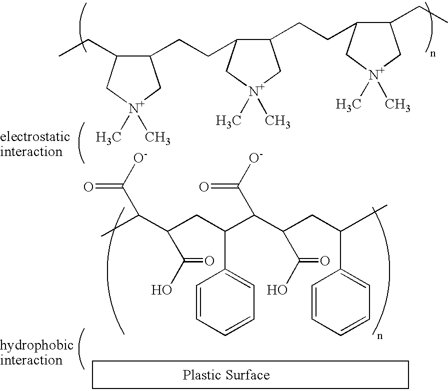

A Harvard 22 syringe pump was used to serially flow fluids through the microfluidic chip while vacuum was used to simultaneously remove the excess fluid from tip of the chip thereby preventing cross contamination of the sheath flow channel, as shown below.

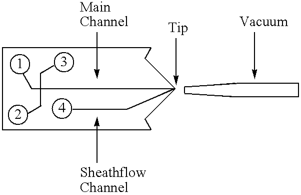

UpChurch Scientific ¼-20 flat bottom fittings were used in conjunction with a custom polycarbonate chip-mount that uses an o-ring pressure seal to connect to the microfluidic chip. Water was continuously flowed through the sheath flow channel (through well 4 at a rate of 20-30 μl/min) throughout all steps of the coating procedure. The main channel of the microfluidic chip was first washed with a 40% aqueous methanol solution followed by drying with vacuum at the tip. A 1% aqueous solution of PMSA was then pumped through the main channel (through wells 1, 2 and 3 at a rate of 15-75 μl/min) for 3 minutes and then the fluidic top was removed and the microfluidic chip was allowed to equilibrate for 10-15 minutes. The PSMA solution was then removed from the wells and the wells and tip were thoroughly rinsed with water. Water was then pumped through the main channel (through wells 1, 2 and 3 at a rate of 15-25 μl/min) for 2-3 minutes, followed by a 0.5% aqueous solution of PDADMAC pumped through the main channel (through wells 1, 2 and 3 at a rate of 15-75 μl/min) for 3 minutes. The fluidic top was removed and the microfluidic chip was allowed to equilibrate for 10-15 minutes. The PDADMAC solution was removed from the wells and the wells and tip were thoroughly rinsed with water. Water was then pumped through the main channel (through wells 1, 2 and 3 at a rate of 15-75 μl/min) for 2-3 minutes. Finally, excess water was removed from all the wells using vacuum; vacuum at the tip removed water from the sheath flow and main channel of microfluidic chip. The microfluidic chip was stored dry until use.

### Example 2

### Preparation of Additional Positively-Charged Coatings

Variations of the bilayer presented in Example 1 are made by substituting for PSMA one of the polymers (or the polymer products resulting from hydrolysis or reaction with other nucleophiles) shown in Table 2.

TABLE 2
Examples of Polymeric Reagents

| Name | Chemical Structure | Source |
|---|---|---|
| Poly(styrene-alt- maleic anhydride) (PSMAA) | 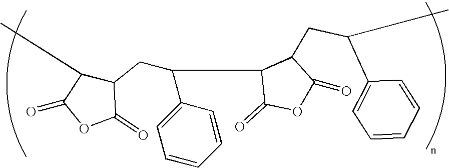 | (MW ˜350,000), available from Aldrich as the partial methyl ester. |
| poly(styrene-alt- maleic acid) (PSMA) | 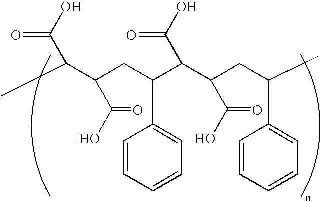 | Made via hydrolysis of PSMAA or available from Aldrich (Mw ˜120,000). |
| Poly(styrene-co- maleic anhydride) | 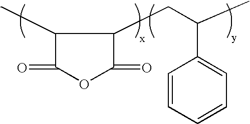 | Aldrich |
| Poly(styrene-co- maleic acid) | 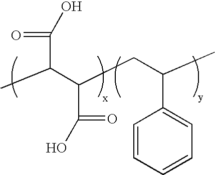 | Made via hydrolysis of poly(styrene-co- maleic anhydride), which may be purchased from Aldrich. |
| Poly(maleic anhydride-1- octadecene | 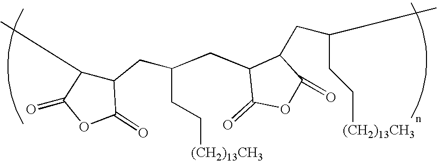 | Polysciences, Inc. |
| Poly(maleic anhydride-1-alt- 1-octadecene | 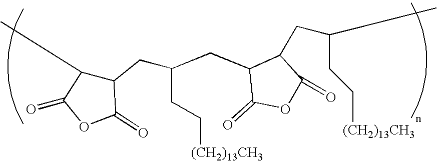 | Aldrich |
| Poly(maleic anhydride-alt-1- tetradecene) | 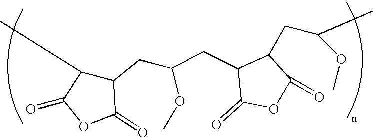 | Aldrich |
| Poly(methyl vinyl ether-alt- maleic anhydride) | 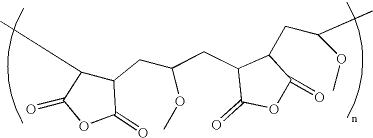 | Aldrich. Polymer is preferably hydrolyzed with a reagent comprising a hydrophobic group. |
| Polyethylene- graft-maleic anhydride | 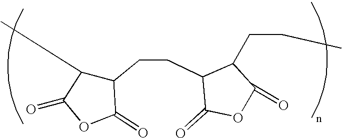 | Aldrich. Polymer is preferably hydrolyzed with a reagent comprising a hydrophobic group. |
| Poly(pyromellitic dianhydride-co- 4,4′-oxydianiline), amic acid solution | 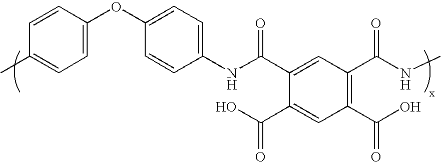 | Aldrich |
| Poly((4,4′- carbonylbis(1,2- benzenedicarboxylic acid))-alt-(4,4′- methylenedianiline) | 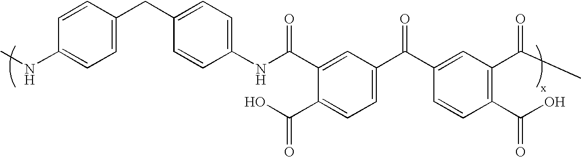 | Aldrich |
| Poly(3,3′,4,4′- benzophenonetet racarboxylic dianhydride-co- 4,4′-oxydianiline/1,3- phenylenediamine), amic acid (solution) | | Aldrich |

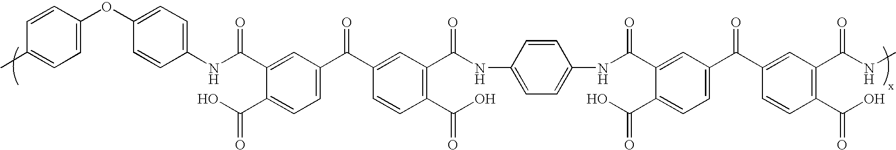

| | | |
|---|---|---|
| Poly(3,3′,4,4′- biphenyltetracarboxylic dianhydride-co-1,4- phenylenediamine), amic acid solution | 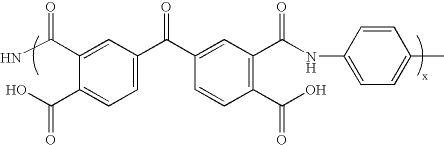 | Aldrich |

### Example 3

### Preparation of Additional Positively-Charged Coatings

Variations of the bilayer presented in Example 1 are made by substituting for PDADMAC one of the polymers (or the polymer products resulting from hydrolysis or reaction with other nucleophiles) shown in Table 3.

TABLE 3
Examples of Polymeric Reagents

| Name | Chemical Structure | Source |
|---|---|---|
| poly(diallyldimethyl- ammonium chloride)²(PDADMAC) | 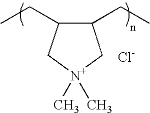 | Available from Aldrich (Mw ˜400,000-500,000) as a 20% solution in water. |
| Poly(3-chloro-2- hydroxypropyl-2- methacryloxyethyl- dimethylammonium chloride) (PCHPMEDMAC) | 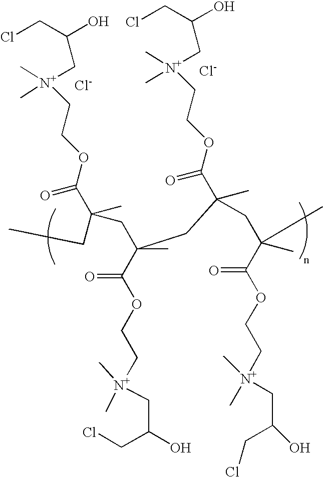 | PolySciences, Inc. |
| Poly(2-hydroxy-3- methacryloxypropyl- trimethylammonium chloride), | 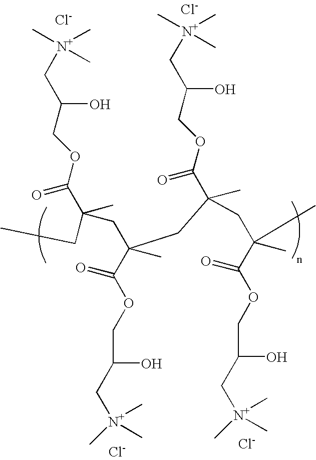 | PolySciences, Inc. |
| Poly(N,N-dimethyl- 3,5-dimethylene piperidinium chloride) | 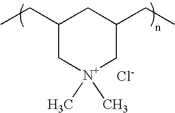 | Scientific Polymer Products |
| Poly(lysine) | 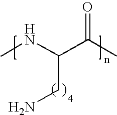 | Sigma |
| Linear polyethyleneimine (1PEI) | 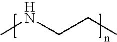 | Made from hydrolysis of Poly(2- ethyl-2-oxazoline) or purchased from PolySciences, Inc. |
| Poly(dimethylamine- co-epichlorohydrin), quaterized | 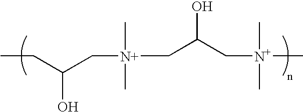 | Scientific Polymer Products |
| Poly(1- vinylpyrrolidone-co-2- dimethylaminoethyl methacrylate), quaternized solution | 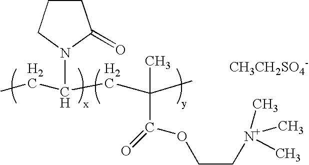 | Aldrich |
| Poly(vinylamine) | 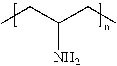 | PolySciences, Inc. |
| Poly(N-methyl vinylamine) | 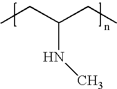 | PolySciences, Inc. |
| Poly(1- vinylpyrrolidone-co-2- dimethylaminoethyl methacrylate | 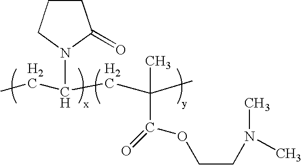 | Aldrich |
| Poly(4-vinyl-1- methylpyridinium bromide | 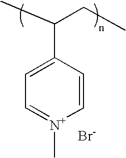 | Polysciences, Inc. |
| Polyallylamine | 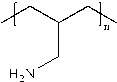 | Polysciences, Inc. |
| Poly(vinyl chloride- co-1-methyl-4- vinylpiperazine) | 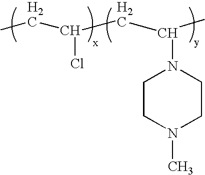 | Aldrich |
| Polyethylenimine, branched | 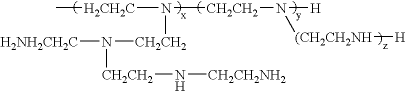 | Aldrich |
| Polyethylenimine, branched 80% ethoxylated | 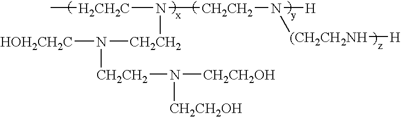 | Aldrich |
| Polyethylenimine, linear | 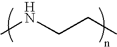 | Polysciences Inc. |
| Gafquat HS-100 a commercial copolymer of MAPTAC:PVP 1:1 | 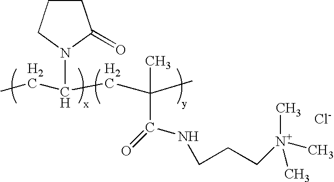 | International Specialty Products |
| Poly(3- methacryloylamino)- propyl]- trimethylammonium chloride). | 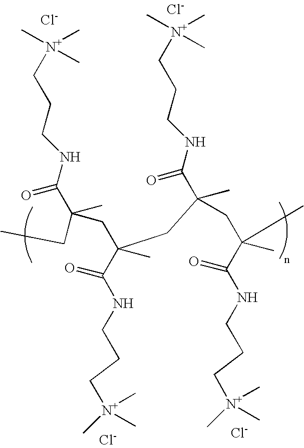 | Synthesized from [3- methacryloylamino)- propyl]- trimethylammonium chloride |
| Poly(2- methacryloyloxy- ethyl]- trimethylammonium chloride) | 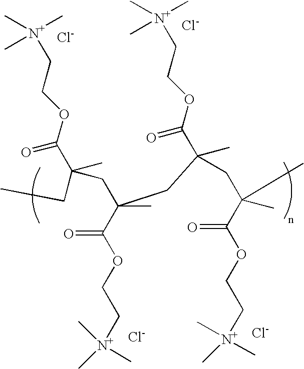 | Synthesized from [2- (methacryloyloxy)- ethyl]- trimethylammonium chloride] |
| Poly(N-[3- (dimethylamino)- propyl]methacrylamide]) | 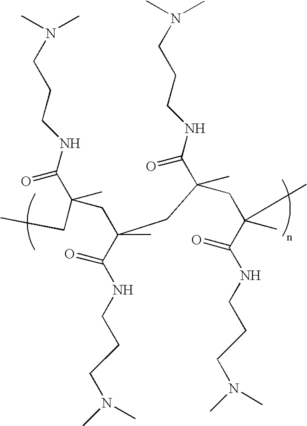 | Synthesized from N- [3-(Dimethylamino)- propyl]methacrylamide] |
| Poly(2- (Dimethylamino)ethyl methacrylate) | 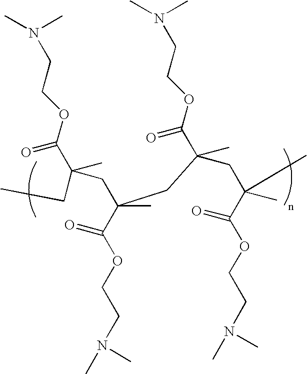 | Synthesized from 2- (Dimethylamino)- ethyl methacrylate |
| Polybrene, Poly(N,N,N′,N′- tetramethyl-N- trimethylenehexa- methylenediammonium dibromide) | 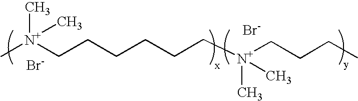 | Sigma |

¹ PDADMAC is available from Aldrich as a 20% w/v solution in water in low, medium or high molecular weights (100,000-200,000; 200,000-350,000; and 400,000-500,000, respectively).

### Example 4

### Preparation of Additional Positively-Charged Coatings

Positively-charged bilayers were prepared by functionalizing or incorporating other functional groups into the PSMA polymer. For example, reaction of PSMAA with ethanolamine produced the following polymer, which was coated onto the hydrophobic surface following the procedure described in Example 1.

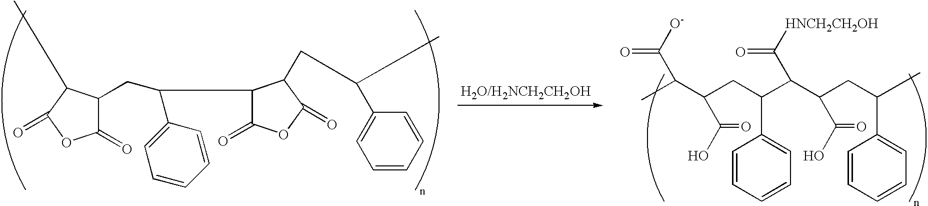

The cationic polymer CHPMEDMAC was activated with a base, such as DBU, and then coated onto the HOCH₂CH₂NH₂-functionalized PSMA layer using the method described in Example 1. The presence of the nucleophile, i.e., the alcohol, in the PSMA layer allows covalent crosslinking with the activated cationic polymer.

### Example 5

### Preparation of Coatings via Functionalized Amphiphilic Polymers

The presence of electrophilic groups such as epoxides or chlorohydrins in the PSMA layer (shown below) allows for covalent crosslinking of cationic polymers that contain nucleophiles, including by way of example only, alcohols or primary amino groups. The carboxylic acid groups of PSMA may also be covalently crosslinked with nucleophiles such as amines or alcohols following reaction with certain activating reagents, including by way of example only, N-(3-dimethylaminopropyl)-N′-ethyl-carbodimide (EDC).

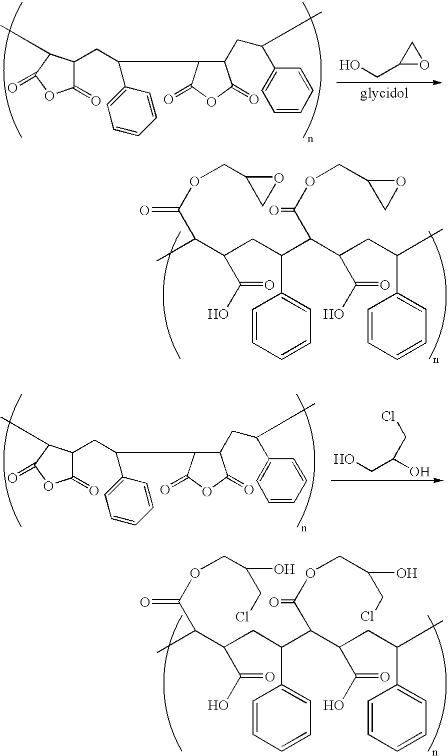

Examples of cationic polymers that may be covalently attached and/or crosslinked to such reactive surfaces are shown below. For example, reaction of PHMAPTAC with glycidol functionalized PSMA produces a coating having the following proposed structure: 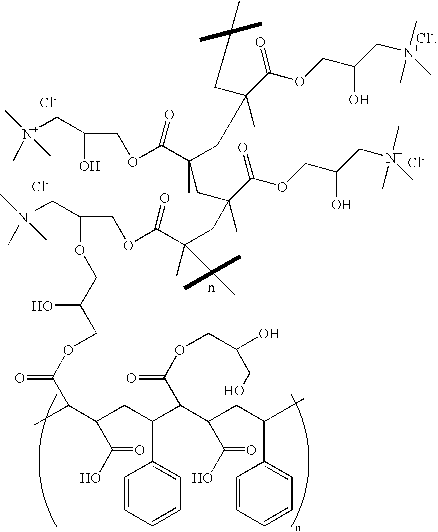
As an additional example, reaction of a co-polymer containing primary and quaternary amino groups with PSMA containing glycidol or chlorohydrin functional groups produces a coating having the following proposed structure:

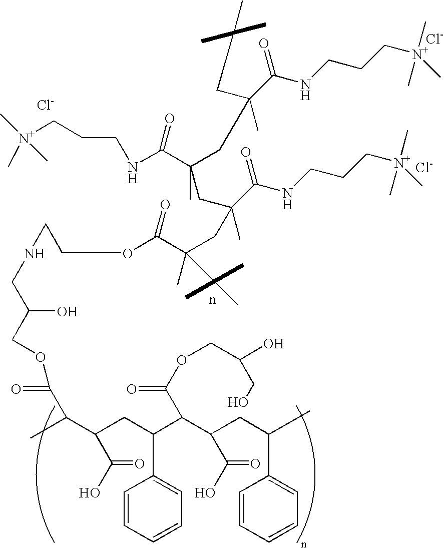

### Example 6

### Preparation of Customized Cationic Polymers

Custom cationic polymers are made via co-polymerization of monomers containing amino groups and monomers containing functional groups that have no overall charge over a pH range of 1-14. Polyethylene glycol methyl ether methacrylate (or other oligoethylene glycol based acrylates), 3-chloro-2-hydroxy-propyl methacrylate, glycidyl methacrylate, [3-(methacryloylamino)propyl]-dimethyl (3-sulfopropyl)ammonium hydroxide, [2-(methacryloyloxy)ethyl]-dimethyl-(3-sulfopropyl)-ammonium hydroxide, 4-acryloxymorpholine, dimethylacrylamide, methacrylamide, are examples of monomers containing functional groups that have no overall charge. 3-methacryloylamino)propyl]-trimethylammonium chloride (MAPTAC), -(methacryloyloxy_ethyl]-trimetylammonium chloride, 2-Aminoethyl methacrylate hydrochloride, 2-(Dimethylamino)ethyl methacrylate and N-[3-(dimethylamino)propyl]methacrylamide] are examples of monomers that contain positive charge at values of pH from 1-10. Examples of the synthesis of homopolymers and co-polymers are shown below.

### Example 6A

### [3-methacryloylamino)propyl]-trimethylammonium chloride (MAPTAC) Polymerization

A 10% w/v solution of ammonium persulfate (APS, NH₄S₂O₈) was prepared by adding 50 mg of ammonium persulfate to 0.5 mL of degassed water. A 5% v/v of MAPTAC (20 mL) was filtered through a 0.22 μm TEFLON syringe filter and degassed overnight in vacuo. To the degassed MAPTAC solution were added TEMED (44 uL) and 140 μL of the 10% solution of APS. The solution was mixed and polymerized in vacuo overnight. The resulting solution turned slightly yellow in color and has a much higher viscosity than the unpolymerized solution.

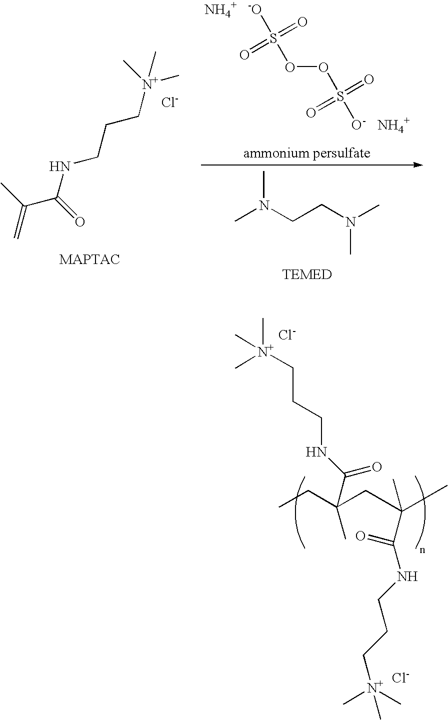

### Example 6B

### Copolymerization of a Mixture of Monomers

A 5% monomer concentration of 2-(methacryloyloxyethyl]-trimethylammonium chloride (TMAEMC 79% w/v of total monomer), 4-acryloylmorpholine (19% w/v of total monomer), and 2-aminoethyl methacrylate (2% w/v of total monomer), was prepared, filtered through a 0.22 μm TEFLON syringe filter and degassed in vacuo overnight. The degassed monomer solution was polymerized using APS and TEMED as described in Example 6A. 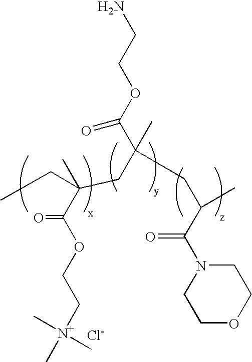
Various cationic polymers were prepared in this manner using a combination of the aforementioned monomers. The charge density of the resulting polymer may be selectively tuned by adjusting the relative concentration of charged and uncharged monomeric subunits.

### Example 7

### Preparation of Coating Using Radical Polymerization

The channels of a microfluidic chip were first washed with an aqueous solution of methanol (40% v/v) for 1 minute and then dry used vacuum. Next, the channels were filled with neat 1,14-tetradecanediol dimethacrylate. After 1 hour the non-adsorbed 1,14-tetradecanediol dimethacrylate was removed using vacuum and the channels were rinsed with an aqueous solution of methanol (40% v/v) for 1 minute and dried using vacuum. Polymerization was performed by pumping an aqueous solution of 0.2% v/v N,N,N,N-tetramethylethylenediamine (TEMED), 0.07% w/v ammonium persulfate (APS) and 5% w/v MAPTAC through the channels for 3 hours. Finally, the chip was washed with water and stored dry until use. See FIG. 8 for a schematic of this coating procedure.

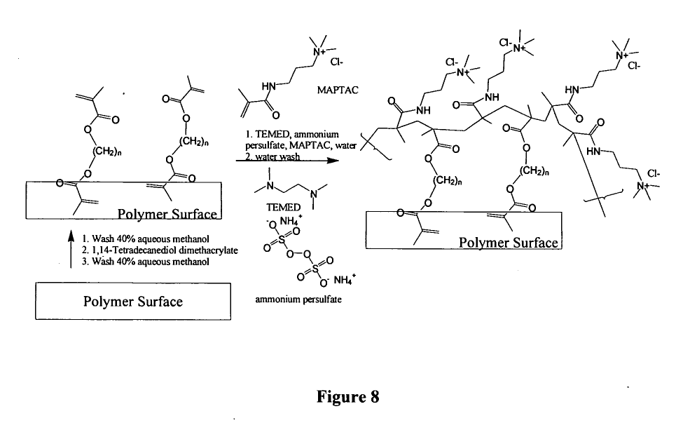

An electrophoresis microfluidic chip, in which the separation channel was coated as described above, was used to separate a mixture of bodipy labeled proteins/peptides. The separation channel was 8 cm long and separation was performed at −450 V/cm in a buffer containing 20% v/v isopropanol and 0.05% v/v formic acid. FIG. 9 is an illustrative plot of the resulting fluorescence intensity vs. time.

### Example 8

### Preparation of Coating by Covalent Attachment

In addition to physically adsorbing a polymer onto a hydrophobic surface, a hydrophilic or amphiphilic polymer may also be covalently attached to the hydrophobic surface; if needed, the hydrophobic surface or the hydrophilic or amphiphilic polymer may require initial activation with an appropriate reagent.

### Example 8A

### Covalent Attachment of a Chlorhydrin Based Polymer to the Surface of Polycarbonate (see FIG. 10A).

Poly(3-chloro-2-hydroxypropyl-2-methacryloxyethyldimethylammonium chloride) was covalently attached to the surface of polycarbonate by application of an aqueous solution of poly(3-chloro-2-hydroxypropyl-2-methacryloxyethyldimethylammonium chloride) (1% w/v) and 1,8-diazabicyclo[5.4.0]undec-7-ene (DBU 5% v/v) for 2 hours. The surface was washed with water and stored until used.

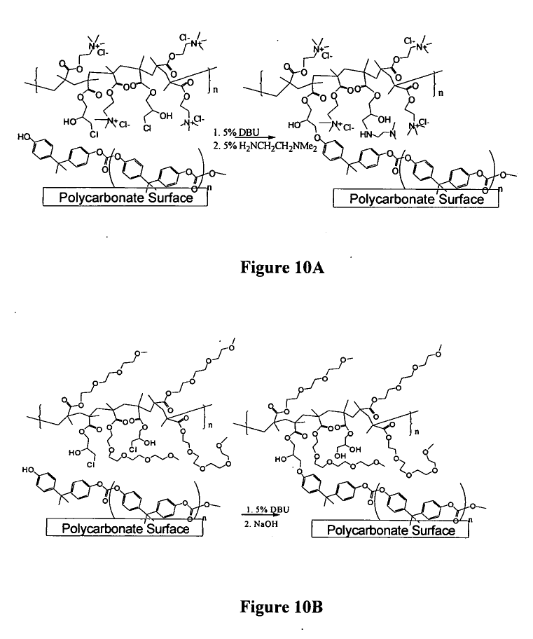

An electrophoresis microfluidic chip, in which the separation channel was coated as described above, was used to separate a mixture of bodipy labeled proteins/peptides. The separation channel was 8 cm long and separation was performed at −300 V/cm in a buffer containing 25% v/v ethanol and 0.1% v/v formic acid. FIG. 11 is an illustrative plot of the resulting fluorescence intensity vs. time.

### Example 8B

### Covalent Attachment of a Chlorhydrin Based Polymer to the Surface of Polycarbonate (see FIG. 10B)

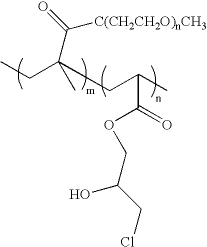

A 5% monomer concentration of 3-chloro-2-hydroxy-propyl methacrylate (CHPMA 5% w/v of total monomer) and poly(ethylene glycol) methyl ether methacrylate (95% w/v of total monomer) was prepared, filtered through a 0.22 μm TEFLON syringe filter and degassed in vacuo overnight. The degassed monomer solution was polymerized using APS and TEMED as described in Example 6A.

Poly(3-chloro-2-hydroxy-propyl methacrylate-co-poly(ethylene glycol) methyl ether methacrylate) was covalently attached to the surface of polycarbonate by application of an aqueous solution of Poly(3-chloro-2-hydroxy-propyl methacrylate-co-poly(ethylene glycol) methyl ether methacrylate) (1% w/v) and 1,8-diazabicyclo[5.4.0]undec-7-ene (DBU 5% v/v) for 8 hours. The surface was washed with water and stored until used.

### Example 9

### Preparation of Coating by Covalent Attachment

The surface of molded PSMAA was exposed to a solution of 0.5% copolymer of 2-(methacryloyloxyethyl]-trimethylammonium chloride (TMAEMC 79% w/v of total monomer), 4-acryloylmorpholine (19% w/v of total monomer), and 2-aminoethyl methacrylate (2% w/v of total monomer) in a pH 11 buffer for 1 hour. Contact angle measurements demonstrated that the resulting surface was hydrophilic.

### Example 10

### Stability Data

Fifty 8 cm microfluidic chips were coated with a PSMA-PDADMAC coating and stored dry in a clean room until use. The electrophoretic separation of a mixture of proteins/peptides that were tagged with a Bodipy fluorophor was measured at various time intervals. In each experiment, three separations were performed on each chip for each of three previously coated chips and for three control chips (coated that day). Graphs of the migration time and theoretical plate number for the Bodipy-labeled ubiquitin and Angiotensin I plotted as a function of time (See FIGS. 21 and 22).

### Example 11

### Preparation of Coating by Solution Swelling (see FIG. 14)

A 10 mL 50% isopropanol solution was prepared by mixing 5 mL of isopropanol with 5 mL of deionized water. 15 mg of (Hydroxypropyl) methyl cellulose (Aldrich) was dissolved in the 50% IPA solution. The solution bottle was agitated on a shaker table overnight until the (Hydroxypropyl) methyl cellulose completely dissolved. The solution should not be vortexed. The coating solution may be stored with closed cap at room temperature.

5 μL of coating solution was added into each of the three reservoirs, sample inlet, sample outlet, and buffer inlet of a PMMA microfluidic chip. Vacuum was applied from the buffer outlet reservoir to draw the coating solution from the other three reservoirs into the channels until all were filled. It is important to watch for blocked channels. The vacuum was applied for an additional 10 min. The coating solution was emptied first from all the reservoirs and then the channels were dried using the vacuum. About 50 μL of deionized water was pushed from the buffer outlet reservoir using a syringe; it takes about 2 min to push through 50 μL of water. Again, it is important to watch for blocked channels. The chip was completely dried with vacuum, and stored dry in a clean box at room temperature.

### Example 12

### Preparation of a Fluorescently-Modified Coating

To a 10% w/v solution of PSMAA (10 ml in anhydrous acetone) was added 2.5 mg of 4,4-difluoro-5,7-dimethyl-4-bora-3a,4a-diaza-s-indacene-3-propionyl ethylenediamine, hydrochloride (Bodipy-amine, Molecular Probes, Eugene Oreg.) and the solution was stirred for 3 hours. Water (1 ml) was added and the reaction was stirred overnight. The resulting solution was added drop wise to 100 ml of 0.1 N sodium hydroxide and then the pH was adjusted to ˜6-7 with 6 N hydrochloric acid. The bodipy labeled PSMA (PSMA-Bodipy) was then dialyzed against 100 mM sodium chloride pH ˜6-7 using a 10 ml Foat-A-Lyzer with a 25 K cutoff from Spectrum laboratories. PSMA-Bodipy was used for formation of the bilayer with PDADMAC as described in Example 1B. FIG. 20 presents a fluorescence image of a microfluidic chip in which the separation channel was coated with PSMA/PDADMAC-Bodipy while the side channel was not coated.

### Example 13

### Preparation of a Fluorescently-Modified Coating

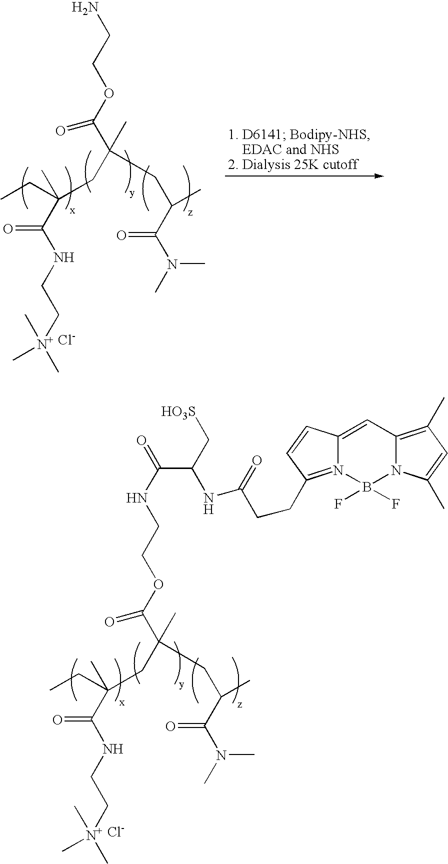

A 5% total monomer concentration of [3-methacryloylamino)propyl]-trimethylammonium chloride (MAPTAC 88% w/v of total monomer), N,N-dimethylmethacrylate (10% w/v of total monomer), and 2-aminoethyl methacrylate (2% w/v of total monomer), was prepared, filtered through a 0.22 um TEFLON syringe filter and degassed in vacuo overnight. The degassed monomer solution was polymerized using APS and TEMED as described in Example 1. To 10 ml of this copolymer solution was added 50 mg of N-hydroxysuccinimide, 100 mg of EDAC and 2.0 mg of N-(4,4-difluoro-5,7-dimethyl-4-bora-3a,4a-diaza-s-indacene-3-propionyl)cysteic acid, succinimidyl ester, triethylammonium salt (Molecular Probes, Eugene, Oreg.). The reaction was allowed to stir over night. The Bodipy labeled cationic polymer (MAPTAC-Bodipy) was then dialyzed against water pH ˜6 using a 10 ml Foat-A-Lyzer with a 25 K cutoff from Spectrum laboratories. MAPTAC-Bodipy was used for formation of the bilayer as a substitute for PDADMAC in the protocol described in Example 1B. FIG. 19 presents a fluorescence image of a microfluidic chip in which the separation channel was coated with PSMA-Bodipy/MAPTAC while the side channel was not coated.

While certain embodiments have been shown and described herein, such embodiments are provided by way of example only. Numerous variations, changes, and substitutions will now occur to those skilled in the art without departing from the devices, compositions and methods described herein. It should be understood that various alternatives to the embodiments of the devices, compositions and methods described herein may be employed equivalently. It is intended that the following claims define the scope of the invention and that methods and structures within the scope of these claims and their equivalents be covered thereby.

We claim:

1. A surface comprising the structure S/A/Z, wherein
S is selected from the group consisting of a hydrophobic surface, a covalently modified hydrophobic surface and a functionalized hydrophobic surface,
A is an amphiphilic region comprising a monolayer of an amphiphilic polymer or a modified amphiphilic polymer, and
Z is a charged region comprising a monolayer of a non-amphiphilic charged polymer or a modified non-amphiphilic charged polymer;
wherein the interaction between S and A comprises hydrophobic interactions and/or covalent bonds, and the interaction between A and Z comprises electrostatic and/or covalent bonds.

2. The surface of claim 1 wherein the amphiphilic polymer or modified amphiphilic polymer is no more than a monolayer.

3. The surface of any of claims 1 or 2, wherein the charged polymer or modified charged polymer is no more than a monolayer.

4. The surface of claim 1 wherein S is a hydrophobic surface comprising a hydrophobic polymer.

5. The surface of claim 4 wherein the amphiphilic polymer or modified amphiphilic polymer is no more than a monolayer.

6. The surface of any of claims 4 or 5, wherein the charged polymer or modified charged polymer is no more than a monolayer.

7. The surface of claim 4 wherein the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof.

8. The surface of claim 4 wherein the hydrophobic surface comprises a mixture or blend of at least two hydrophobic polymers.

9. The surface of claim 1 wherein S is a modified hydrophobic surface comprising a modified hydrophobic polymer.

10. The surface of claim 9 wherein the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

11. The surface of claim 7 wherein the hydrophobic polymer is a methacrylate polymer.

12. The surface of claim 7 wherein the hydrophobic polymer is polycarbonate.

13. The surface of claim 10 wherein the hydrophobic polymer is a modified methacrylate polymer.

14. The surface of claim 10 wherein the hydrophobic polymer is modified polycarbonate.

15. The surface of any of claims 13-14 wherein the modification is a covalent modification.

16. The surface of any of claims 13-14 wherein the modification is a partial modification.

17. A method for forming the modified hydrophobic polymer of claim 9 comprising exposing a hydrophobic polymer surface with a nucleophile.

18. A method for forming the modified hydrophobic polymer of claim 9 comprising exposing a hydrophobic polymer surface with an electrophile.

19. The method of any of claims 17 or 18 wherein the exposing step is sufficient to partially modify the hydrophobic polymer surface.

20. The method of any of claims 17 or 18 wherein the hydrophobic polymer surface is a methacrylate surface.

21. The method of any of claims 17 or 18 wherein the hydrophobic polymer surface is a polycarbonate surface.

22. The surface of claim 1, wherein A comprises an amphiphilic polymer.

23. The surface of claims 1, wherein A comprises a modified amphiphilic polymer.

24. The surface of claim 22 wherein the amphiphilic polymer comprises a moiety selected from the group consisting of an aryl, an alkyl, and a halogenated alkyl.

25. The surface of claim 23 wherein the modified amphiphilic polymer comprises a moiety selected from the group consisting of an aryl, an alkyl, and a halogenated alkyl.

26. The surface of claim 22 wherein the amphiphilic polymer comprises polystyrene units.

27. The surface of claim 23 wherein the modified amphiphilic polymer comprises polystyrene units.

28. The surface of claim 22 wherein the amphiphilic polymer comprises positively charged moieties.

29. The surface of claim 22 wherein the amphiphilic polymer comprises negatively charged moieties.

30. The surface of claim 22 wherein the amphiphilic polymer comprises maleic anhydride units.

31. The surface of claim 22 wherein the amphiphilic polymer is derived from maleic anhydride units.

32. A method of making the amphiphilic region of claim 1 comprising reacting a non-amphiphilic polymer with at least one nucleophile to form an amphiphilic polymer.

33. The method of claim 32 wherein the nucleophile is a charged nucleophile.

34. The method of claim 32 wherein the nucleophile is a neutral nucleophile.

35. The method of claim 32 further comprising reacting the non-amphiphilic polymer with an additional nucleophile.

36. The method of claim 32 wherein at least a portion of the non-amphiphilic polymer is in contact with S prior to the reacting step.

37. The method of claim 32 further comprising exposing the amphiphilic polymer to S.

38. The method of claim 37 wherein the exposing step is prior to the reacting step.

39. The method of claim 37 wherein the exposing step is after the reacting step.

40. The method of claim 37 wherein the exposing step is simultaneous with the reacting step.

41. The method of any of claims 38-40 further comprising reacting the amphiphilic polymer with an additional reagent thereby forming a modified amphiphilic surface.

42. The method of claim 32 wherein the non-amphiphilic polymer comprises maleic anhydride units.

43. The method of any of claims 38-40 wherein S is a hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof.

44. The method of any of claims 38-40 wherein S is a modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

45. The method of claim 43 wherein the hydrophobic polymer is a methacrylic polymer.

46. The method of claim 43 wherein the hydrophobic polymer is a polycarbonate polymer.

47. The surface of any of claims 1, 22 or 23, wherein Z is a non-amphiphilic charged polymer.

48. The surface of any of claims 1, 22 or 23, wherein Z is a modified non-amphiphilic charged polymer.

49. The surface of claim 28 wherein Z comprises negatively-charged moieties.

50. The surface of claim 29 wherein Z comprises positively-charged moieties.

51. The surface of claim 50 wherein the positively-charged moieties are quarternary amines.

52. The surface of claim 47 wherein the molecular weight of Z is greater than 20,000 atomic mass units.

53. The surface of claim 48 wherein the molecular weight of Z is greater than 20,000 atomic mass units.

54. A method for making the charged region of claim 1 comprising exposing a surface comprising the structure S/A to non-amphiphilic charged polymer.

55. The method of claim 54 further comprising reacting the non-amphiphilic charged polymer with a reagent thereby forming a modified non-amphiphilic charged polymer.

56. The method of claim 55 wherein the exposing step is prior to the reacting step.

57. A surface comprising the structure S/P/R, wherein
S is selected from the group consisting of a hydrophobic surface, a covalently modified hydrophobic surface, and a functionalized hydrophobic surface,
P is a functionalized region comprising a monolayer of a linkable hydrophobic polymer or a modified linkable hydrophobic polymer, and
R is a charged region comprising a monolayer of a linkable charged hydrophilic polymer or a modified linkable charged hydrophilic polymer;
wherein the interaction between S and P comprises hydrophobic interactions and/or covalent bonds, and the interaction between P and R comprises covalent bonds, and/or electrostatic bonds, and/or hydrophobic interactions.

58. The surface of claim 57 wherein the linkable hydrophobic polymer or the modified linkable hydrophobic polymer is no more than a monolayer.

59. The surface of any of claim 57 or 58, wherein the linkable charged hydrophilic polymer or modified linkable charged hydrophilic polymer is no more than a monolayer.

60. The surface of claim 57 wherein S is a hydrophobic surface comprising of a hydrophobic polymer.

61. The surface of claim 60 wherein the linkable hydrophobic polymer or the modified linkable hydrophobic polymer is no more than a monolayer.

62. The surface of any of claims 60 or 61, wherein the linkable charged hydrophilic polymer or modified linkable charged hydrophilic polymer is no more than a monolayer.

63. The surface of claim 60 wherein the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof.

64. The surface of claim 60 wherein the hydrophobic surface comprises a mixture or blend of at least two hydrophobic polymers.

65. The surface of claim 57 wherein S is a modified hydrophobic surface comprising of a modified hydrophobic polymer.

66. The surface of claim 65 wherein the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

67. The surface of claim 63 wherein the hydrophobic polymer is a methacrylate polymer.

68. The surface of claim 63 wherein the hydrophobic polymer is polycarbonate.

69. The surface of claim 66 wherein the hydrophobic polymer is a modified methacrylate polymer.

70. The surface of claim 66 wherein the hydrophobic polymer is modified polycarbonate.

71. The surface of any of claims 69 or 70 wherein the modification is a covalent modification.

72. The surface of any of claims 69 or 70 wherein the modification is a partial modification.

73. A method for forming the modified hydrophobic polymer of claim 65 comprising exposing a hydrophobic polymer surface with a nucleophile.

74. A method for forming the modified hydrophobic polymer of claim 65 comprising exposing a hydrophobic polymer surface with an electrophile.

75. The method of any of claims 73 or 74 wherein the exposing step is sufficient to partially modify the hydrophobic polymer surface.

76. The method of any of claims 73 or 74 wherein the hydrophobic polymer surface is a methacrylate surface.

77. The method of any of claims 73 or 74 wherein the hydrophobic polymer surface is a polycarbonate surface.

78. The surface of claim 57, wherein P comprises a linkable hydrophobic polymer.

79. The surface of claim 57, wherein P comprises a modified linkable hydrophobic polymer.

80. The surface of claim 78 wherein the linkable hydrophobic polymer comprises a moiety selected from the group consisting of an aryl, an alkyl, and a halogenated alkyl.

81. The surface of claim 78 wherein the linkable hydrophobic polymer comprises a moiety selected from the group consisting of a vinyl and a substituted vinyl.

82. The surface of claim 80 wherein the linkable hydrophobic polymer further comprises a moiety selected from the group consisting of a vinyl and a substituted vinyl.

83. The surface of claim 79 wherein the modified linkable hydrophobic polymer comprises a moiety selected from the group consisting of an aryl, an alkyl, and a halogenated alkyl.

84. The surface of claim 79 wherein the modified linkable hydrophobic polymer comprises a moiety selected from the group consisting of a vinyl, and a substituted vinyl.

85. The surface of claim 83 wherein the modified linkable hydrophobic polymer further comprises a moiety selected from the group consisting of a vinyl, and a substituted vinyl

86. The surface of claim 78 wherein the linkable hydrophobic polymer comprises poly(1,14-tetradecanediol dimethacrylate) units.

87. The surface of claim 79 wherein the modified linkable hydrophobic polymer comprises poly(1,14-tetradecanediol dimethacrylate) units.

88. A method of making the functionalized region of claim 57 comprising reacting a non-linkable hydrophobic polymer with at least one nucleophile to form the linkable hydrophobic polymer.

89. The method of claim 88 wherein the nucleophile comprises a moiety selected from the group consisting of a vinyl and a substituted vinyl.

90. The method of claim 88 further comprising reacting the non-linkable hydrophobic polymer with an additional nucleophile.

91. The method of claim 88 wherein at least a portion of the non-linkable hydrophobic polymer is in contact with S prior to the reacting step.

92. The method of claim 88 further comprising exposing the non-linkable hydrophobic polymer to S prior to the reacting step.

93. The method of claim 88 further comprising exposing the non-linkable hydrophobic polymer to S simultaneous with the reacting step.

94. A method of making the functionalized region of claim 57 comprising exposing reactive monomeric units of the linkable hydrophobic polymer to S.

95. The method of claim 94 further comprising polymerizing the reactive units thereby forming the linkable hydrophobic polymer on S.

96. The method of any of claims 92, 93 or 95 further comprising reacting the linkable hydrophobic polymer with an additional reagent thereby forming a modified linkable hydrophobic surface.

97. The method of any of claims 92, 93 or 95 wherein S is a hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof.

98. The method of any of claims 92-93 wherein S is a modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

99. The method of claim 97 wherein the hydrophobic polymer is a methacrylic polymer.

100. The method of claim 97 wherein the hydrophobic polymer is a polycarbonate polymer.

101. The surface of any of claims 57, 78 or 79, wherein R is a linkable charged hydrophilic polymer.

102. The surface of any of claims 57, 78 or 79, wherein R is a modified linkable charged hydrophilic polymer.

103. A method of making the charged region of claim 57 comprising exposing the linkable charged hydrophilic polymer to the linkable hydrophobic polymer on S, and reacting the linkable charged hydrophilic polymer with at least a portion of the linkable hydrophobic polymer on S.

104. A method of making the charged region of claim 57, comprising exposing monomeric units of the linkable charged hydrophilic polymer to the linkable hydrophobic polymer on S reacting the monomeric units of the linkable charged hydrophilic polymer with at least a portion of the linkable hydrophobic polymer on S.

105. A method of making the charged region of claim 57 comprising exposing the modified reactive charged hydrophilic polymer to the reactive hydrophobic polymer on S reacting the modified linkable charged hydrophilic polymer with at least a portion of the linkable hydrophobic polymer on S.

106. A method of making the charged region of claim 57, comprising exposing monomeric units of the modified linkable charged hydrophilic polymer to the linkable hydrophobic polymer on S polymerizing the monomeric units of the modified linkable charged hydrophilic polymer with at least a portion of the linkable hydrophobic polymer on S.

107. The surface of claim 101 wherein R comprises negatively-charged moieties.

108. The surface of claim 101 wherein R comprises positively-charged moieties.

109. The surface of claim 101 wherein R comprises moieties with charge equal to zero.

110. The surface of claim 102 wherein R comprises negatively-charged moieties.

111. The surface of claim 102 wherein R comprises positively-charged moieties.

112. The surface of claim 102 wherein R comprises moieties with charge equal to zero.

113. The surface of claim 108 wherein the positively-charged moieties are quarternary amines.

114. The surface of claim 111 wherein the positively-charged moieties are quarternary amines.

115. The surface of claim 101 wherein the molecular weight of R is greater than 20,000 atomic mass units.

116. The surface of claim 102 wherein the molecular weight of R is greater than 20,000 atomic mass units.

117. A surface comprising the structure S/N, wherein
S is selected from the group consisting of a hydrophobic surface, a covalently modified hydrophobic surface, and a functionalized hydrophobic surface,
N is a hydrophilic region comprising a monolayer of neutral hydrophilic polymer or a modified neutral hydrophilic polymer;
wherein the interaction between S and N comprises physical entrapment of at least a portion of N in S.

118. The surface of claim 117 wherein the neutral hydrophilic polymer or a modified neutral hydrophilic polymer is no more than a monolayer.

119. The surface of claim 117 wherein S is a hydrophobic surface comprising a hydrophobic polymer.

120. The surface of claim 119 wherein the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof.

121. The surface of claim 119 wherein the hydrophobic surface comprises a mixture or blend of at least two hydrophobic polymers.

122. The surface of claim of claim 117 wherein S is a modified hydrophobic surface comprising a modified hydrophobic polymer.

123. The surface of claim 122 wherein the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

124. The surface of claim 119 wherein the hydrophobic polymer is a methacrylate polymer.

125. The surface of claim 119 wherein the hydrophobic polymer is polycarbonate.

126. The surface of claim 122 wherein the hydrophobic polymer is a modified methacrylate polymer.

127. The surface of claim 122 wherein the hydrophobic polymer is modified polycarbonate.

128. The surface of claim 122 wherein the modification is a covalent modification.

129. The surface of claim 122 wherein the modification is a partial modification.

130. A method for forming the modified hydrophobic polymer of claim 122 comprising exposing a hydrophobic polymer surface with a nucleophile.

131. A method for forming the modified hydrophobic polymer of claim 122 comprising exposing a hydrophobic polymer surface with an electrophile.

132. The method of any of claims 130 or 131 wherein the exposing step is sufficient to partially modify the hydrophobic polymer surface.

133. The method of any of claims 130 or 131 wherein the hydrophobic polymer surface is a methacrylate surface.

134. The method of any of claims 130 or 131 wherein the hydrophobic polymer surface is a polycarbonate surface.

135. The surface of any of claims 117, 118, 119 or 122, wherein N comprises a neutral hydrophilic polymer.

136. The surface of any of claims 117, 118, 119 or 122, wherein N comprises a modified neutral hydrophilic polymer.

137. The surface of claim 135 wherein the neutral hydrophilic polymer is selected from the group consisting of a poly(ethylene glycol) derivative, a poly(ethylene oxide) derivative, a cellulose derivatives, and combinations thereof.

138. The surface of claim 136 wherein the modified hydrophilic polymer is selected from the group consisting of a modified poly(ethylene glycol) derivative, a modified poly(ethylene oxide) derivative, a modified cellulose derivatives, and combinations thereof.

139. The surface of claim 137 wherein the neutral hydrophilic polymer comprises poly(ethylene glycol) units.

140. The surface of claim 137 wherein the neutral hydrophilic polymer comprises poly(ethylene oxide) units.

141. The surface of claim 137 wherein the neutral hydrophilic polymer comprises hydroxypropylmethyl cellulose units.

142. The surface of claim 138 wherein the modified neutral hydrophilic polymer comprises modified poly(ethylene glycol) units.

143. The surface of claim 138 wherein the modified neutral hydrophilic polymer comprises modified poly(ethylene oxide) units.

144. The surface of claim 138 wherein the modified neutral hydrophilic polymer comprises modified hydroxypropylmethyl cellulose units.

145. A method of making the neutral region of claim 117 comprising swelling the hydrophobic surface with a solvent, and exposing the swollen hydrophobic surface to the neutral hydrophilic polymer.

146. The method of claim 145 further comprising drying the swollen hydrophobic surface sufficient to entrap at least a portion of the neutral hydrophilic polymer within at least a portion of the hydrophobic surface.

147. The method of claim 145 further comprising reacting the neutral hydrophilic polymer with a reagent to form a modified neutral hydrophilic polymer.

148. A surface comprising the structure S/C, wherein
S is selected from the group consisting of a hydrophobic surface, a covalently modified hydrophobic surface, and a functionalized hydrophobic surface,
C is a hydrophilic region comprising a monolayer of a linkable hydrophilic polymer or a linkable modified hydrophilic polymer;
wherein the interaction between S and C comprises covalent attachment of at least a portion of C onto S.

149. The surface of claim 148 wherein the linkable hydrophilic polymer or a linkable modified hydrophilic polymer is no more than a monolayer.

150. The surface of claim 148 wherein S is a hydrophobic surface comprising a hydrophobic polymer.

151. The surface of claim 150 wherein the hydrophobic polymer is selected from the group consisting of a polyolefin, a styrene polymer, a halogenated hydrocarbon polymer, a vinyl polymer, an acrylic polymer, an acrylate polymer, a methacrylic polymer, a methacrylate polymer, a polyester, an anhydride polymer, a polyacrylamide, a cyclo-olefin polymer, a polysiloxane, a polycarbonate, and copolymers thereof.

152. The surface of claim 150 wherein the hydrophobic surface comprises a mixture or blend of at least two hydrophobic polymers.

153. The surface of claim of claim 148 wherein S is a modified hydrophobic surface comprising a modified hydrophobic polymer.

154. The surface of claim 153 wherein the modified hydrophobic polymer is selected from the group consisting of a modified polyolefin, a modified styrene polymer, a modified halogenated hydrocarbon polymer, a modified vinyl polymer, a modified acrylic polymer, a modified acrylate polymer, a modified methacrylic polymer, a modified methacrylate polymer, a modified polyester, a modified anhydride polymer, a modified polyacrylamide, a modified cyclo-olefin polymer, a modified polysiloxane, a modified polycarbonate, and modified copolymers thereof.

155. The surface of claim 151 wherein the hydrophobic polymer is a methacrylate polymer.

156. The surface of claim 151 wherein the hydrophobic polymer is polycarbonate.

157. The surface of claim 151 wherein the hydrophobic polymer is poly(styrene-co-maleic anhydride).

158. The surface of claim 154 wherein the hydrophobic polymer is a modified methacrylate polymer.

159. The surface of claim 154 wherein the hydrophobic polymer is a modified polycarbonate.

160. The surface of claim 151 wherein the hydrophobic polymer is a modified poly(styrene-co-maleic anhydride).

161. The surface of any of claims 158-160 wherein the modification is a covalent modification.

162. The surface of any of claims 158-160 wherein the modification is a partial modification.

163. A method for forming the modified hydrophobic polymer of claim 153 comprising exposing a hydrophobic polymer surface with a nucleophile.

164. A method for forming the modified hydrophobic polymer of claim 153 comprising exposing a hydrophobic polymer surface with an electrophile.

165. The method of any of claims 163 or 164 wherein the exposing step is sufficient to partially modify the hydrophobic polymer surface.

166. The method of any of claims 163 or 164 wherein the hydrophobic polymer surface is a methacrylate surface.

167. The method of any of claims 163 or 164 wherein the hydrophobic polymer surface is a polycarbonate surface.

168. The surface of any of claims 148, 149, 150 or 153, wherein C comprises a linkable hydrophilic polymer.

169. The surface of any of claims 148, 149, 150 or 153, wherein C comprises a linkable modified hydrophilic polymer.

170. The surface of claim 168, wherein the linkable hydrophilic polymer comprises positively charged moieties.

171. The surface of claim 168, wherein the linkable hydrophilic polymer comprises negatively charged moieties.

172. The surface of claim 168, wherein the linkable hydrophilic polymer is neutral.

173. The surface of claim 169, wherein the linkable modified hydrophilic polymer comprises positively charged moieties.

174. The surface of claim 169, wherein the linkable modified hydrophilic polymer comprises negatively charged moieties.

175. The surface of claim 169, wherein the linkable modified hydrophilic polymer is neutral.

176. The surface of claim 168 wherein the linkable hydrophilic polymer is selected from the group consisting of a polysaccharide, a polyether, a poly(alcohol), a polyamide, a protein, a polyacrylonitrile, a zwitterionic polymer, a poly(acrylic acid), a polystyrenesulfonic acid, a polyvinylphosphonic acid, a poly(glutamic acid), a poly(aspartic acid), a poly(anilinesulfonic acid), a poly(3-sulfopropyl methacrylate), a polyethanolesulfonate, a heparin, a polyamine, a polyethyleneimine, a polyallylamine, a poly(N-methyl vinylamine, a poly(vinylamine), a poly(lysine), a poly(3-chloro-2-hydroxypropyl-2-methacryloxyethyl-dimethylammonium chloride), and copolymers thereof.

177. The surface of claim 169 wherein the linkable modified hydrophilic polymer is selected from the group consisting of a modified polysaccharide, a modified polyether, a modified poly(alcohol), a modified polyamide, a modified protein, a modified polyacrylonitrile, a modified zwitterionic polymer, a modified poly(acrylic acid), a modified polystyrenesulfonic acid, a modified polyvinylphosphonic acid, a modified poly(glutamic acid), a modified poly(aspartic acid), a modified poly(anilinesulfonic acid), a modified poly(3-sulfopropyl methacrylate), a modified polyethanolesulfonate, a modified heparin, a modified polyamine, a modified polyethyleneimine, a modified polyallylamine, a modified poly(N-methyl vinylamine, a modified poly(vinylamine), a modified poly(lysine), a modified poly(3-chloro-2-hydroxypropyl-2-methacryloxyethyl-dimethylammonium chloride), and copolymers thereof.

178. The surface of claim 168 wherein the linkable hydrophilic polymer is a poly(ethyeneimine).

179. The surface of claim 169 wherein the linkable modified hydrophilic polymer is poly(N-methyl vinylamine).

180. A method of making the hydrophilic region of claim 148 comprising:
exposing the hydrophobic surface or the modified hydrophobic surface with a hydrophilic polymer or a modified hydrophilic polymer comprised of linkable moieties; and
reacting the linkable moieties with at least a portion of the hydrophobic surface or the modified hydrophobic surface.

181. The method of claim 180 wherein the linkable unit is a nucleophile.

182. The method of claim 180 wherein the linkable unit is an electrophile.

183. The method of claim 180 wherein the linkable unit is chlorohydrin.

184. A microfluidic chip for mass spectrometric analysis comprising:
a microfluidic body layer formed with a plurality of fluid reservoirs;
at least one separation channel and/or at least one side channel that are formed along a length of the microfluidic body layer in fluid communication with at least one fluid reservoir;
wherein at least one of the separation channels and/or side channels comprises a charged polymer monolayer coated on a hydrophobic surface; and
a cover plate for enclosing the separation channel and the side channel to provide a stable electrospray from the microfluidic chip.

185. The microfluidic chip as recited in claim 184, wherein the side channel provides electrical contact to the separation channel.

186. The microfluidic chip as recited in claim 184, wherein the side channel provides sheath flow.

187. The microfluidic chip as recited in claim 184, wherein the charged coating of the side channel is a negatively charged coating, and the separation channel includes a positively charged coating.

188. The microfluidic chip of claim 187, wherein each of the charged coatings are produced using the method of claim 17.

189. The microfluidic chip of claim 187, wherein each of the charged coatings are produced using the method of claim 18.

190. The microfluidic chip of claim 187, wherein each of the charged coatings are produced using the method of claim 32.

191. The microfluidic chip of claim 187, wherein each of the charged coatings are produced using the method of claim 54.

192. The microfluidic chip as recited in claim 184, wherein the charged coating of the side channel is a negatively charged coating, and the separation channel is without a coating.

193. The microfluidic chip of claim 192, wherein the negatively charged coating is produced using the method of claim 17.

194. The microfluidic chip of claim 192, wherein the negatively charged coating is produced using the method of claim 18.

195. The microfluidic chip of claim 192, wherein the negatively charged coating is produced using the method of claim 32.

196. The microfluidic chip of claim 192, wherein the negatively charged coating is produced using the method of claim 54.

197. The microfluidic chip as recited in claim 184, wherein the charged coating of the side channel is a negatively charged coating, and the separation channel includes a neutral uncharged coating.

198. The microfluidic chip of claim 197, wherein the negatively charged coating is produced using the method of claim 17.

199. The microfluidic chip of claim 197, wherein the negatively charged coating is produced using the method of claim 18.

200. The microfluidic chip of claim 197, wherein the negatively charged coating is produced using the method of claim 32.

201. The microfluidic chip of claim 197, wherein the negatively charged coating is produced using the method of claim 54.

202. The microfluidic chip of claim 197, wherein the neutral uncharged coating is produced using the method of claim 130.

203. The microfluidic chip of claim 197, wherein the neutral uncharged coating is produced using the method of claim 131.

204. The microfluidic chip of claim 197, wherein the neutral uncharged coating is produced using the method of claim 145.

205. The microfluidic chip as recited in claim 184 wherein the charged coating of the side channel is a positively charged coating, and the separation channel includes a negatively charged coating.

206. The microfluidic chip of claim 205, wherein each of the charged coatings are produced using the method of claim 17.

207. The microfluidic chip of claim 205, wherein each of the charged coatings are produced using the method of claim 18.

208. The microfluidic chip of claim 205, wherein each of the charged coatings are produced using the method of claim 32.

209. The microfluidic chip of claim 205, wherein each of the charged coatings are produced using the method of claim 54.

210. The microfluidic chip as recited in claim 184, wherein the charged coating of the side channel is a positively charged coating, and the separation channel is without a coating.

211. The microfluidic chip of claim 210, wherein the positively charged coating is produced using the method of claim 17.

212. The microfluidic chip of claim 210, wherein the positively charged coating is produced using the method of claim 18.

213. The microfluidic chip of claim 210, wherein the positively charged coating is produced using the method of claim 32.

214. The microfluidic chip of claim 210, wherein the positively charged coating is produced using the method of claim 54.

215. The microfluidic chip as recited in claim 184, wherein the charged coating of the side channel is a positively charged coating, and the separation channel includes a neutral uncharged coating.

216. The microfluidic chip of claim 215, wherein the positively charged coating is produced using the method of claim 17.

217. The microfluidic chip of claim 215, wherein the positively charged coating is produced using the method of claim 18.

218. The microfluidic chip of claim 215, wherein the positively charged coating is produced using the method of claim 32.

219. The microfluidic chip of claim 215, wherein the positively charged coating is produced using the method of claim 54.

220. The microfluidic chip of claim 215, wherein the neutral uncharged coating is produced using the method of claim 130.

221. The microfluidic chip of claim 215, wherein the neutral uncharged coating is produced using the method of claim 131.

222. The microfluidic chip of claim 215, wherein the neutral uncharged coating is produced using the method of claim 145.

223. The microfluidic chip as recited in claim 184, wherein the side channel is without a coating, and the separation channel includes a positively charged coating.

224. The microfluidic chip of claim 223, wherein the positively charged coating is produced using the method of claim 17.

225. The microfluidic chip of claim 223, wherein the positively charged coating is produced using the method of claim 18.

226. The microfluidic chip of claim 223, wherein the positively charged coating is produced using the method of claim 32.

227. The microfluidic chip of claim 223, wherein the positively charged coating is produced using the method of claim 54.

228. The microfluidic chip as recited in claim 184, wherein the side channel is without a coating, and the separation channel includes a negatively charged coating.

229. The microfluidic chip of claim 228, wherein the negatively charged coating is produced using the method of claim 17.

230. The microfluidic chip of claim 228, wherein the negatively charged coating is produced using the method of claim 18.

231. The microfluidic chip of claim 228, wherein the negatively charged coating is produced using the method of claim 32.

232. The microfluidic chip of claim 228, wherein the negatively charged coating is produced using the method of claim 54.

233. The microfluidic chip as recited in claim 184, wherein the side channel is includes a neutral coating, and the separation channel includes a positively charged coating.

234. The microfluidic chip of claim 233, wherein the neutral uncharged coating is produced using the method of claim 130.

235. The microfluidic chip of claim 233, wherein the neutral uncharged coating is produced using the method of claim 131.

236. The microfluidic chip of claim 233, wherein the neutral uncharged coating is produced using the method of claim 145.

237. The microfluidic chip of claim 233, wherein the positively charged coating is produced using the method of claim 17.

238. The microfluidic chip of claim 233, wherein the positively charged coating is produced using the method of claim 18.

239. The microfluidic chip of claim 233, wherein the positively charged coating is produced using the method of claim 32.

240. The microfluidic chip of claim 233, wherein the positively charged coating is produced using the method of claim 54.

241. The microfluidic chip as recited in claim 184, wherein the side channel is includes a neutral coating, and the separation channel includes a negatively charged coating.

242. The microfluidic chip of claim 241, wherein the neutral uncharged coating is produced using the method of claim 130.

243. The microfluidic chip of claim 241, wherein the neutral uncharged coating is produced using the method of claim 131.

244. The microfluidic chip of claim 241, wherein the neutral uncharged coating is produced using the method of claim 145.

245. The microfluidic chip of claim 241, wherein the negatively charged coating is produced using the method of claim 17.

246. The microfluidic chip of claim 241, wherein the negatively charged coating is produced using the method of claim 18.

247. The microfluidic chip of claim 241, wherein the negatively charged coating is produced using the method of claim 32.

248. The microfluidic chip of claim 241, wherein the negatively charged coating is produced using the method of claim 54.

249. The microfluidic chip as recited in claim 184, further comprising:
a plurality of electrodes positioned in each fluid reservoir to apply voltages to impart movement of materials within the separation channel and the side channel.

250. The microfluidic chip as recited in claim 184, wherein the cover plate extends beyond the microfluidic body layer to form an open-ended distal tip portion at which the separation channel and the side channel terminate to provide an electrospray ionization tip that directs a stable electrospray from the microfluidic chip.

251. The microfluidic chip as recited in claim 184, wherein at least a portion of the open-ended distal tip portion is covered with a hydrophilic material.

252. The microfluidic chip as recited in claim 184, wherein the tapered end portion of the microfluidic body layer includes a tapered end formed along a substantially flat truncated portion of the tapered end portion.

253. A microfluidic chip for electrospray ionization comprising:
a channel plate formed with a separation channel and at least two side channels that are each in fluid communication with at least one fluid reservoir included within the channel plate, and wherein at least one side channel includes a charged coating;
and a covering plate for substantially enclosing the non-intersecting fluid channels formed on the channel plate, wherein the covering plate includes an overhang that extends beyond the channel plate to provide an electrospray tip that includes an open-tip region at which each of the non-intersecting fluid channels terminate.

254. The microfluidic chip as recited in claim 253, further comprising:
a syringe in fluid communication with a side channel to provide sheath flow.

255. The microfluidic chip as recited in claim 253, wherein the charged coating of the side channel includes positively or negatively charged molecules.

256. The microfluidic chip as recited in claim 253, wherein the charged coating of the side channel includes negatively charged molecules, and wherein the separation channel has a charged coating that includes positively charged molecules.

257. The microfluidic chip as recited in claim 253, wherein the charged coating of the side channel is a positively charged coating, and the separation channel is without a coating.

258. The microfluidic chip as recited in claim 253, wherein the charged coating of the side channel is a positively charged coating, and the separation channel includes a neutral uncharged coating.

259. The microfluidic chip of any of claims 184 or 253 fabricated by pressure molding poly(styrene-co-maleic anhydride).,54
